(19) United States
(12) Patent Application Publication
(10) Pub. No.: US 2024/0180737 A1
(43) Pub. Date: Jun. 6, 2024

(54) CONDUITS INCLUDING AT LEAST ONE CONDUIT POROUS MATERIAL

(71) Applicant: PUREWICK CORPORATION, Covington, GA (US)

(72) Inventor: Camille Rose Newton, Bonsall, CA (US)

(21) Appl. No.: 18/553,625

(22) PCT Filed: Apr. 6, 2022
(86) PCT No.: PCT/US2022/023594

§ 371 Date: Oct. 2, 2023

Related U.S. Application Data
(60) Provisional application No. 63/172,975, filed on Apr. 9, 2021.

(51) Int. Cl. A61F 5/44; A61F 5/451

(52) U.S. Cl. CPC A61F 5/4404; A61F 5/451

(57) ABSTRACT

Embodiments disclosed herein include conduits including at least one conduit porous material, fluid collection assemblies and systems including the same, and methods of using and forming the same. An example conduit includes at least one wall at least partially defining at least an inlet, and outlet downstream from the inlet, and a passageway extending from the inlet to the outlet. The conduit also includes at least one conduit porous material disposed in the passageway. The conduit porous material may at least partially occupy the passageway and may extend along at least a portion of a length of the conduit measured from the inlet to the outlet. The conduit may be configured for use in a fluid collection system for collecting one or more bodily fluids (e.g., urine, amniotic fluid, blood, etc.).

### CROSS-REFERENCE TO RELATED APPLICATION(S)

This application claims priority to U.S. Patent Provisional Application No. 63/172,975 filed on Apr. 9, 2021, the disclosure of which is incorporated herein, in its entirety, by this reference.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Further, conduits used in association with urinary catheters may be prone to kinking and collapsing.

Thus, users and manufacturers of fluid collection assemblies continue to seek new and improved devices, systems, and methods to collect urine.

### SUMMARY

Embodiments disclosed herein include conduits including at least one conduit porous material, fluid collection assemblies and systems including the same, and methods of using and forming the same. In an embodiment, a conduit for use in a fluid collection system for collecting one or more bodily fluids is disclosed. The conduit includes at least one wall at least partially defining at least an inlet, an outlet, and a passageway extending from the inlet to the outlet. The conduit also includes at least one conduit porous material disposed in at least a portion of the passageway.

In an embodiment, a fluid collection system is disclosed. The fluid collection system includes a fluid collection assembly. The fluid collection assembly includes a fluid impermeable barrier defining at least a chamber, at least one opening, and a fluid outlet. The fluid collection assembly also includes at least one assembly porous material disposed in the chamber. The fluid collection system also includes the conduit in fluid communication with the chamber. The conduit includes at least one wall at least partially defining at least an inlet, an outlet, and a passageway extending from the inlet to the outlet. The conduit also includes at least one conduit porous material disposed in at least a portion of the passageway.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a cross-sectional schematic of a conduit, according to an embodiment.

FIGS. 2A to 2D are cross-sectional schematics of different conduits taken along a plane that is perpendicular to a longitudinal axis of the conduits, according to different embodiments.

FIG. 3 is a schematic cross-sectional view of a portion of a conduit, according to an embodiment.

FIGS. 4A and 4B illustrate a method of forming a conduit that includes a conduit porous material extending outwardly from the passageway (illustrated in FIG. 4C), according to an embodiment.

FIG. 4C is a cross-sectional schematic of the conduit when the first portion of the conduit porous material is folded back onto the walls, according to an embodiment.

FIG. 5 is an cross-sectional schematic of a conduit that includes a first portion of the conduit porous material extending from the passageway of the conduit, according to an embodiment.

FIGS. 6A and 6B illustrate a method of forming a conduit that includes a conduit porous material extending outwardly from the passageway (not shown, obscured), according to an embodiment.

FIG. 7A is an isometric view of a portion of a fluid collection system that includes a fluid collection assembly that is in fluid communication with a conduit, according to an embodiment.

FIG. 7B is a cross-sectional schematic of the fluid collection system taken along plane 7B-7B shown in FIG. 7A, according to an embodiment.

FIG. 8 is a cross-sectional schematic of a portion of a fluid collection system that includes a conduit in fluid communication with a fluid collection assembly, according to an embodiment.

FIG. 9 is a cross-sectional schematic of a portion of a fluid collection system, according to an embodiment.

FIG. 10 is a cross-sectional schematic of a portion of a fluid collection system, according to an embodiment.

FIG. 11 is a cross-sectional schematic of a portion of a fluid collection system that includes a conduit in fluid communication with a fluid collection assembly, according to an embodiment.

FIG. 12 is a cross-sectional schematic of a portion of a fluid collection system that includes a conduit in fluid communication with a fluid collection assembly, according to an embodiment.

FIG. 13 is a cross-sectional schematic of a portion of a fluid collection system that includes a conduit in fluid communication with a fluid collection assembly, according to an embodiment.

FIG. 14 is a cross-sectional schematic of a portion of a fluid collection system that includes a conduit in fluid communication with a fluid collection assembly, according to an embodiment.

FIG. 15A is an isometric view of a portion of a fluid collection system that includes a conduit in fluid communication with a fluid collection assembly, according to an embodiment.

FIG. 15B is a cross-sectional schematic of the fluid collection assembly taken along plane 15B-15B shown in FIG. 15A, according to an embodiment.

FIG. 16 is a cross-sectional schematic of a portion of a fluid collection system that includes a conduit in fluid communication with a fluid collection assembly, according to an embodiment.

FIG. 17 is a cross-sectional schematic of a portion of a fluid collection system that includes a conduit in fluid communication with a fluid collection assembly, according to an embodiment.

FIG. 18 is a cross-sectional schematic of a portion of a fluid collection system that includes a conduit in fluid communication with a fluid collection assembly, according to an embodiment.

FIG. 19 is a cross-sectional schematic of a portion of a fluid collection system that includes a conduit in fluid communication with a fluid collection assembly, according to an embodiment.

FIG. 20 is a cross-sectional schematic of a portion of a fluid collection system that includes a conduit in fluid communication with a fluid collection assembly, according to an embodiment.

FIG. 21 is a block diagram of a fluid collection system for fluid collection, according to an embodiment.

### DETAILED DESCRIPTION

Embodiments disclosed herein include conduits having at least one conduit porous material, fluid collection assemblies and systems including the same, and methods of using and forming the same. An example conduit includes at least one wall at least partially defining at least an inlet, and outlet downstream from the inlet, and a passageway extending from the inlet to the outlet. The conduit also includes at least one conduit porous material disposed in the passageway. The conduit porous material may at least partially occupy the passageway and may extend along at least a portion of a length of the conduit measured from the inlet to the outlet. The conduit may be configured for use in a fluid collection system for collecting one or more bodily fluids (e.g., urine, amniotic fluid, blood, etc.).

The conduits including the conduit porous material may be an improvement over a hollow conduit that does not include the conduit porous material. The hollow conduits may be used in a fluid collection system, such as a fluid collection system that includes a vacuum source. The fluid collection system may include a fluid collection assembly that is configured to receive one or more bodily fluids from a patient. The hollow conduit may be in fluid communication with both the fluid collection assembly and the vacuum source such that a vacuum pressure applied to the hollow conduit by the vacuum source may remove the bodily fluids from the fluid collection assembly. The hollow conduit is configured to prevent collapse thereof when the vacuum pressure is applied thereto since the collapse of the hollow conduit may inhibit the removal of the bodily fluids from the fluid collection assembly. The hollow conduit is configured to prevent collapse thereof by forming the hollow conduit from at least one material exhibiting a Young's modulus (e.g., modulus of elasticity) and/or a thickness that prevent the collapse of the hollow conduit when the vacuum pressure is applied thereto. For example, the hollow conduit may be formed from transparent polyvinyl chloride and the wall thereof may exhibit a thickness (e.g., measured parallel to the diameter thereof) that is greater than about 1.5 mm. It is noted that the hollow conduit may be formed from the same material and/or exhibit the same thickness discussed above even when the hollow conduit is used in a fluid collection system that does not include a vacuum source to prevent a collapse thereof when a mass is disposed thereon.

The hollow conduit may exhibit a limited flexibility when the hollow conduit is configured to prevent the collapse thereof. The limited flexibility of the hollow conduits may create several issues. In an example, the hollow conduits may exhibit a limited amount of bending without kinking due the limited flexibility thereof. Similar to the collapsing of the hollow conduit, kinking the hollow conduit inhibits the bodily fluids from being removed from the fluid collection assembly to which the hollow conduit is attached. The limited amount of bending of the hollow conduit may limit the locations where the conduit may be placed and may require longer lengths. In an example, the limited flexibility of the hollow conduit may make conforming the fluid collection assembly to the region about the urethral opening of the patient (e.g., an individual using the fluid collection assembly) difficult. For instance, the hollow conduit may be at least partially disposed within the fluid collection assembly. The fluid collection assembly may be bent to conform to the shape of the urethral opening which may minimize bodily fluids from leaking from the fluid collection assembly. However, the limited flexibility of the hollow conduit may at least one of resist bending of the fluid collection assembly, may kink when the fluid collection assembly is bent, or cause the fluid collection assembly to straighten or otherwise unbend.

As previously discussed, the conduits that include at least one conduit porous material disposed in passageway thereof resolve at least some of the issues of the hollow conduit discussed above. In an example, the conduit porous material provides support to the wall of the conduit, thereby decreasing the likelihood that the conduit collapses when the vacuum pressure is applied thereto. As such, the conduits disclosed herein may be formed from a material exhibiting a Young's modulus that is less than and/or a thickness that is less than the hollow conduit. The lower Young's modulus and/or thickness of the conduits disclosed herein allows such conduits to exhibit a flexibility that is greater than the hollow conduit. Further, even if the conduits disclosed herein exhibit a flexibility that is comparable to the hollow conduits, the conduit porous material provides support to the wall of the conduit such that the conduit is less likely to kink when bent compared to the hollow conduit. The increased flexibility and/or kinking resistance of the conduits that include at least one conduit porous material may also allow such conduits to be more comfortable for a patient compared to the hollow conduits.

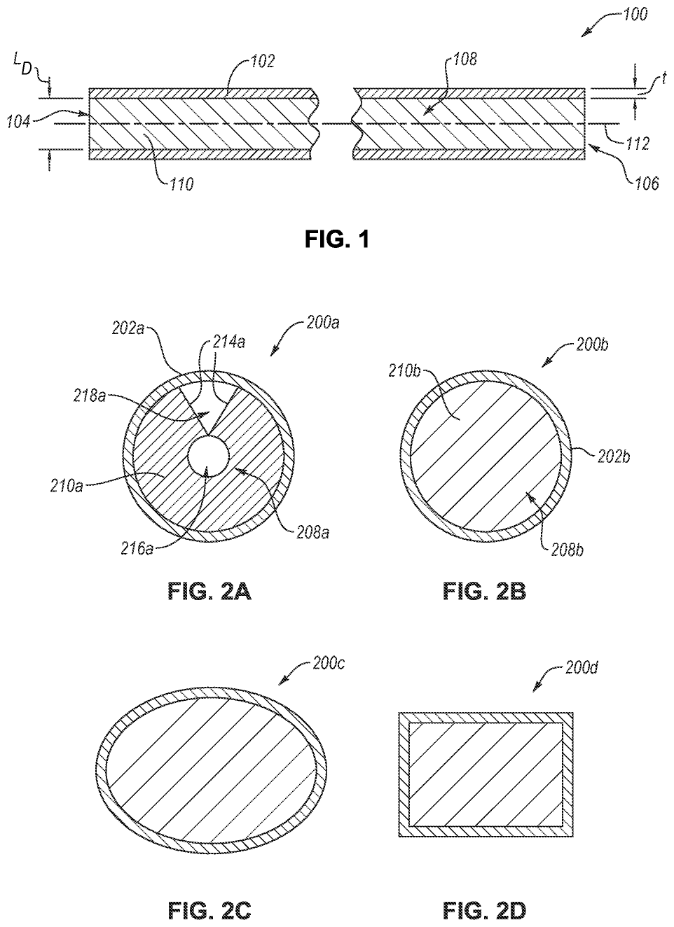

FIG. 1 is a cross-sectional schematic of a conduit 100, according to an embodiment. The conduit 100 includes at least one wall 102. The wall 102 defines at least an inlet 104, and outlet 106 downstream from the inlet 104, and a passageway 108 extending from the inlet 104 to the outlet 106. The inlet 104 may be configured to be connected to, disposed in a chamber of, or otherwise in fluid communication with (e.g., via another conduit) a fluid collection assembly (not shown). The outlet 106 is configured to be connected to or otherwise in fluid communication with (e.g., via another conduit or a fluid storage container) a vacuum source (not shown). The passageway 108 forms a fluid flow path that may remove bodily fluids from a fluid collection assembly (not shown) and deposit the removed bodily fluids in a fluid storage container. The conduit 100 also includes at least one conduit porous material 110 disposed in and at least partially occupying the passageway 108. The conduit porous material 110 provides a matrix through which the bodily fluids may flow (e.g., via capillary action) and provides support to the wall 102 thereby inhibiting collapse or kinking of the conduit 100.

The wall 102 may be formed from any suitable fluid impermeable material. In an embodiment, the wall 102 may be formed from a material that is conventionally used to form hollow conduits. In such an embodiment, the fluid impermeable wall 102 may be formed from polyvinyl chloride. In an embodiment, the wall 102 may be formed from one or more materials exhibiting a Young's modulus that is less than polyvinyl chloride. The wall 102 may be formed from a material exhibiting a Young's modulus that is less than polyvinyl chloride since the conduit porous material 110 provides support to the wall 102. Examples of such materials include polyethylene (e.g., low density polyethylene, high density polyethylene, ultrahigh molecular weight polyethylene), polypropylene, polytetrafluoroethylene, nitrile, nylon, ethylene vinyl acetate, a thermoplastic elastomer, or combinations thereof. In an embodiment, the wall 102 may be formed from a material exhibiting a Young's modulus that is greater the polyvinyl chloride. In such an embodiment, the wall 102 may exhibit a thickness that is less than a hollow conduit which allows the conduit 100 to exhibit a flexibility that is less than or equal to a hollow conduit that is formed from polyvinyl chloride. In an embodiment, the wall 102 may be at least partially formed from a fluid impermeable tape. In such an embodiment, the tape may be adhesively attached to the conduit porous material 110.

The wall 102 may exhibit a thickness t measured perpendicular to the longitudinal axis 112 of the conduit 100. The thickness of the wall 102 may be selected based on the desired flexibility of the conduit 100, the strength of the conduit porous material 110, and the Young's modulus of the material(s) that form the wall 102.

In an embodiment, the wall 102 may exhibit a thickness t that is comparable to or greater than a thickness of a hollow conduit conventionally used in fluid collection systems, such as a thickness t that is about 1.5 mm or more, about 1.6 mm or more, about 1.7 mm or more, about 1.8 mm or more, about 1.9 mm or more, about 2 mm or more, about 2.25 mm or more, about 2.5 mm or more, about 3 mm or more, or in ranges of about 1.5 mm to about 1.7 mm, about 1.6 mm to about 1.8 mm, about 1.7 mm to about 1.9 mm, about 1.8 mm to about 2 mm, about 1.9 mm to about 2.25 mm, about 2 mm to about 2.5 mm, or about 2.25 mm to about 3 mm. The wall 102 may exhibit a thickness that is comparable to or greater than a hollow conduit because at least one of the conduit porous material 110 inhibits kinking of the conduit 100 compared to the hollow conduit or the wall 102 may be formed from a material exhibiting a Young's modulus that is less than polyvinyl chloride. Selecting the wall 102 to exhibit a thickness t that is comparable to or greater than the hollow conduit allows at least one of the conduit 100 to have larger vacuum pressures applied thereto without collapsing, the wall 102 to be formed from materials exhibiting a Young's modulus that is significantly less than polyvinyl chloride (i.e., the wall 102 may be formed from a greater variety of materials), the conduit porous material 110 to be formed from relatively flimsy material(s) (e.g., a material exhibiting a relatively low yield strength, relatively low Young's modulus, and/or a relatively high porosity) such that the conduit porous material 110 does not provide much support to the wall 102, the wall 102 may be formed from a hollow conduit that is commonly used and readily available, or the conduit 100 to be used with inlets and outlets that are configured to be used with the hollow conduits.

In an embodiment, the wall 102 may exhibit a thickness t that is less than the hollow conduit. For example, the wall 102 may exhibit a thickness that is about 0.05 mm or less, about 0.1 mm or less, about 0.15 mm or less, about 0.2 mm or less, about 0.25 mm or less, about 0.3 mm or less, about 0.35 mm or less, about 0.4 mm or less, about 0.45 mm or less, about 0.5 mm or less, about 0.6 mm or less, about 0.7 mm or less, about 0.8 mm or less, about 0.9 mm or less, about 1 mm or less, about 1.1 mm or less, about 1.2 mm or less, about 1.3 mm or less, about 1.4 mm or less, about 1.5 mm or less, or in ranges of about 0.05 mm to about 0.15 mm, about 0.1 mm to about 0.2 mm, about 0.15 mm to about 0.25 mm, about 0.2 mm to about 0.3 mm, about 0.25 mm to about 0.35 mm, about 0.3 mm to about 0.4 mm, about 0.35 mm to about 0.45 mm, about 0.4 mm to about 0.5 mm, about 0.45 mm to about 0.6 mm, about 0.5 mm to about 0.7 mm, about 0.6 mm to about 0.8 mm, about 0.7 mm to about 0.9 mm, about 0.8 mm to about 1 mm, about 0.9 mm to about 1.1 mm, about 1 mm to about 1.2 mm, about 1.1 mm to about 1.3 mm, about 1.2 mm to about 1.4 mm, or about 1.3 mm to about 1.5. The wall 102 disclosed herein may be able to exhibit such small thicknesses because of the support provided from the conduit porous material 110 to the wall 102, even when the wall 102 may be formed from material(s) that exhibit a Young's modulus that is less than polyvinyl chloride.

In an embodiment, the wall 102 may be formed from a film. As used herein, the wall 102 is a “film” when the thickness t of the wall 102 is less than 0.75 mm, more particularly less than about 0.5 mm, or more particularly less than about 0.3 mm. The flexibility of the conduit 100 significantly increases, compared to a hollow conduit, when the wall 102 is a film. For example, the wall 102 substantially prevents kinky and presents little to no resistance to bending of the fluid collection assembly when the wall 102 is a film.

As previously discussed, the wall 102 defines a passageway 108 and at least a portion of the passageway 108 is occupied by the at least one conduit porous material 110. The conduit porous material 110 may provide support to the wall 102 to inhibit collapse and kinking of the conduit 100 when a vacuum pressure is applied to the conduit 100 and when the conduit 100 is bent, respectively. The conduit porous material 110 may also direct the bodily fluids that are pulled into the conduit 100 (e.g., via the vacuum pressure or capillary action) towards the outlet 106. The conduit porous material 110 may define a plurality of interconnected pores through which the bodily fluids may flow.

The conduit porous material 110 may include any suitable conduit porous material. In an example, the conduit porous material 110 may be formed from a fabric, such as a silk, linen, or cotton gauze. In an example, the conduit porous material 110 may be formed from a porous polymer (e.g., nylon, polyester, polyurethane, polyethylene, polypropylene, etc.) structure or an open cell foam, spun nylon fiber, a natural material (e.g., cotton, wool, silk, or combinations thereof), compression gauze, paper, terry cloth, pumice, any other suitable material, or combinations thereof. In an example, the conduit porous material 110 may be formed from spun nylon fibers. In an example, the conduit porous material 110 may be formed from a non-woven material, such as at least one of carded webs, needle punched webs, air laid webs, spunlaced webs, vertical lapped nonwoven fabrics, horizontal lapped nonwoven fabrics, or crossed lapped nonwoven fabrics. The nonwoven fabric may be formed from fibers that include at least one of polyester, polypropylene, polyurethane, polyolefin, polycarbonate, polyvinyl chloride, polyacrylic, nylon, other synthetic fibers, one or more natural fibers (e.g., low grade cotton waste), hollow fibers, or combinations thereof. Such nonwoven fabrics may exhibit a high porosity which allows the bodily fluids to flow therein while also providing sufficient support to the wall 102 to prevent collapse and kinking of the conduit 100. Examples of non-woven materials are disclosed in U.S. Provisional Patent Application No. 63/134,754 filed on Jan. 7, 2021, the disclosure of which is incorporated herein, in its entirety, by this reference.

The conduit porous material 110 may exhibit a percent porosity that is about 20% or greater, about 30% or greater, about 40% or greater, about 50% or greater, about 60% or greater, about 65% or greater, about 70% or greater, about 75% or greater, about 80% or greater, about 85% or greater, about 90% or greater, about 95% or greater, or in ranges of about 20% to about 40%, about 30% to about 50%, about 40% to about 60%, about 50% to about 65%, about 60% to about 70%, about 65% to about 75%, about 70% to about 80%, about 75% to about 85%, about 80% to about 90%, about 85% to about 95%, or about 90% to about 99%. Generally, decreasing the percent porosity of the conduit porous material 110 may increase the amount of support that the conduit porous material 110 provides to the wall 102. In other words, decreasing the percent porosity of the conduit porous material 110 may decrease the likelihood that the conduit 100 collapses when the vacuum pressure is applied thereto or kinks. However, decreasing the percent porosity of the conduit porous material 110 also decreases the flow rate of bodily fluids flowing through the conduit 100. As such, the percent porosity of the conduit porous material 110 may be selected by balancing the ability of the conduit porous material 110 to provide support to the wall 102 while also allowing the bodily fluids to flow therein.

The conduit porous material 110 at least partially occupies the cross-sectional area of the passageway 108, wherein the cross-sectional area of the passageway 108 is taken along a plane that is perpendicular to the longitudinal axis 112 of the conduit 100. The quantity of the cross-sectional area of the passageway 108 that is occupied by the conduit porous material 110 may depend on the method that is used to form the conduit 100. In an embodiment, the conduit porous material 110 substantially completely occupies the cross-sectional area of the passageway 108. In such an embodiment, the conduit 100 may be formed, for example, by co-extruding the wall 102 and the conduit porous material 110 or disposing the wall 102 directly on the conduit porous material 110. The wall 102 may be directly disposed on the conduit porous material 110, for example, when the wall 102 is tape that is adhesively disposed on the conduit porous material 110 or the wall 102 coats an exterior surface of the conduit porous material 110. It is noted that the wall 102 may extend partially into the conduit porous material 110 when the wall 102 is formed as a coating on the conduit porous material 110. In an embodiment, the conduit porous material 110 only partially occupies the cross-sectional area of the passageway 108 (e.g., the conduit porous material 110 exhibits a cross-sectional area that is less than the cross-sectional area of the passageway 108). In such an embodiment, the conduit 100 may be formed, for example, by inserting the conduit porous material 110 into a hollow conduit. The conduit porous material 110 that is inserted into the hollow conduit may exhibit a cross-sectional area that is less than the cross-sectional area of the hollow conduit to minimize friction caused by the conduit porous material 110 contacting the surfaces of the hollow conduit during insertion. It is noted that the conduit porous material 110 may substantially occupy the cross-sectional area of the passageway 108 when the wall 102 is formed (e.g., wrapped) around the conduit porous material 110 or the conduit porous material 110 is inserted into the passageway 108 of a hollow conduit (such an insertion may be more difficult than if the conduit porous material 110 exhibited a cross-sectional area that is less than the passageway 108). It is noted that the conduit 100 may be formed using methods other than those disclosed above, such as disposing the conduit porous material 110 in a hollow conduit and then heat shrinking the hollow conduit or coating the conduit porous material 110 with a fluid impermeable material that forms the wall 102.

In an embodiment, the conduit 100 may be configured such that the conduit 100 is unlikely to collapse when exposed to an expected vacuum pressure. As used herein, the vacuum pressure refers to the gauge pressure, that is, the pressure differential between a location external to and spaced from the passageway 108 and the fluid collection assembly to which the conduit 100 is attached and a location within the passageway. During use, the vacuum pressure applied to the passageway 108 may be about 1 kPa to about 40 kPa depending on the vacuum source that is fluidly coupled to the fluid collection assembly 1040. As such, the wall 102 and the porous material 110 may be selected to be able to withstand such vacuum pressures of about 1 kPa to about 5 kPa, about 2.5 kPa to about 7.5 kPa, about 5 kPa to about 10 kPa, about 7.5 kPa to about 12.5 kPa, about 10 kPa to about 15 kPa, about 12.5 kPa to about 17.5 kPa, about 15 kPa to about 20 kPa, about 17.5 kPa to about 22.5 kPa, about 20 kPa to about 25 kPa, about 22.5 kPa to about 27.5 kPa, about 25 kPa to about 30 kPa, about 27.5 kPa to about 32.5 kPa, about 30 kPa to about 35 kPa, about 32.5 kPa to about 37.5 kPa, or about 35 kPa to about 40 kPa. In some embodiments, the vacuum pressure may be greater than about 40 kPa.

The ability of the conduit 100 to resist collapse when the vacuum pressure is applied to the passageway 108 depends on several factors. Generally, increasing the Young's modulus of the material that forms the wall 102, increasing the Young's modulus of the material that forms the conduit porous material 110, increasing the thickness of the wall 102, increasing the density of the conduit porous material 110, and decreasing the percent porosity of the conduit porous material 110 allows a greater vacuum pressure to be applied to the passageway 108 without significantly increasing the likelihood that the conduit 100 collapses. However, increasing the Young's modulus of the material that forms the wall 102, increasing the Young's modulus of the material that forms the conduit porous material 110, increasing the thickness of the wall 102, increasing the density of the conduit porous material 110, and decreasing the void space in the passageway 108 decreases the flexibility of the conduit 100. As such, selecting the composition of the wall 102, the composition of the conduit porous material 110, the thickness of the wall 102, the density of the conduit porous material 110, and the quantity of void space may be selected by balancing the need to prevent the vacuum pressure from collapsing the conduit 100 while increasing the flexibility of the conduit 100.

The conduit porous material 110 may be selected to exhibit a density of about 5 kg/m³ to about 10 kg/m³, about 7.5 kg/m³ to about 12.5 kg/m³, about 10 kg/m³ to about 15 kg/m³, about 12.5 kg/m³ to about 17.5 kg/m³, about 15 kg/m³ to about 20 kg/m³, about 17.5 kg/m³ to about 22.5 kg/m³, about 20 kg/m³ to about 25 kg/m³, about 22.5 kg/m³ to about 27.5 kg/m³, about 25 kg/m³ to about 30 kg/m³, about 27.5 kg/m³ to about 32.5 kg/m³, about 30 kg/m³ to about 35 kg/m³, about 32.5 kg/m³ to about 37.5 kg/m³, about 35 kg/m³ to about 37.5 kg/m³, about 35 kg/m³ to about 40 kg/m³, about 37.5 kg/m³ to about 42.5 kg/m³, about 40 kg/m³ to about 45 kg/m³, about 42.5 kg/m³ to about 47.5 kg/m³, or about 45 kg/m³ to about 50 kg/m³. Generally, increasing the density of the conduit porous material 110 decreases the likelihood that the conduit 100 collapses when the vacuum pressure is applied thereto. However, increasing the density of the conduit porous material 110 also decreases the flow rate of the bodily fluids flowing therein and decreases the flexibility of the conduit 100. As such, the density of the conduit porous material 110 may be selected by balancing the need to prevent the vacuum pressure from collapsing the conduit 100 while increasing the flexibility of the conduit 100. The density of the conduit porous material 110 may also be selected based on the Young's modulus and the thickness of the wall 102, wherein increasing the Young's modulus and/or thickness of the wall 102 allows the conduit porous material 110 to exhibiting a lower density, and vice versa. It is noted that the void space in the passageway 108 depends, at least in part, on the density of the conduit porous material 110 and whether the porous material 110 occupies substantially all of the cross-sectional area of the passageway 108.

Generally, the average person discharges urine at a rate of about 6 ml/s to about 50 ml/s, such as at a rate of about 10 ml/s to about 25 ml/s. The rate at which the person urinate may vary, such as based on the size of the person and the age of the person. The conduit porous material 110 may be selected to exhibit a flow rate that is comparable to the rate at which the average person discharges urine to prevent oversaturation of the fluid collection assembly to which the conduit 100 is attached with bodily fluids which may cause leaks. For example, the conduit porous material 110 may be selected to exhibit a flow rate that is greater than about 6 ml/s, greater than about 10 ml/s, greater than about 20 ml/s, greater than about 30 ml/s, greater than about 40 ml/s, greater than about 50 ml/s, or in ranges of about 6 ml/s to about 10 ml/s, about 8 ml/s to about 12 ml/s, about 10 ml/s to about 15 ml/s, about 12.5 ml/s to about 17.5 ml/s, about 15 ml/s to about 20 ml/s, about 17.5 ml/s to about 22.5 ml/s, about 20 ml/s to about 25 ml/s, about 22.5 ml/s to about 27.5 ml/s, about 25 ml/s to about 30 ml/s, about 27.5 ml/s to about 35 ml/s, about 30 ml/s to about 40 ml/s, about 35 ml/s to about 45 ml/s, or about 40 ml/s to about 50 ml/s. As used herein, the flow rate may refer to the flow rate of the bodily fluids in the conduit porous material 110 when the conduit porous material 110 is at least one of saturated with the bodily fluids, not saturated with the bodily fluids, any of the vacuum pressures disclosed herein are applied to the passageway 108, or when no vacuum pressure is applied to the passageway 108 (e.g., the bodily fluids flow only due to wicking and/or gravity).

The conduit 100 is illustrated with a break between the inlet 104 and the outlet 106. The break indicates that the conduit 100 may exhibit any length. In an example, the conduit 100 may exhibit a length that is about 1 cm or greater, about 15 cm or greater, about 30 cm or greater, about 45 cm or greater, about 60 cm or greater, about 75 cm or greater, about 90 cm or greater, about 105 cm or greater, about 120 cm or greater, about 150 cm or greater, about 180 cm or greater, 210 cm or greater, about 240 cm or greater, about 265 cm or greater, about 300 cm or greater, or in ranges of about 1 cm to about 30 cm, about 15 cm to about 45 cm, about 30 cm to about 60 cm, about 45 cm to about 75 cm, about 60 cm to about 90 cm, about 75 cm to about 105 cm, about 90 cm to about 120 cm, about 105 cm to about 150 cm, about 120 cm to about 180 cm, about 150 cm to about 210 cm, about 180 cm to about 240 cm, about 210 cm to about 265 cm, or about 240 cm to about 300 cm. For example, the conduit 100 may exhibit a length of about 1 cm to about 15 cm when the conduit 100 forms a bendable elbow connector between a hollow conduit and another hollow conduit or a fluid collection assembly, a length of about 10 cm to about 40 cm when the conduit 100 is predominately disposed only in a fluid collection assembly, or a length greater than about 35 cm when the conduit 100 extends a significantly distance from the fluid collection assembly.

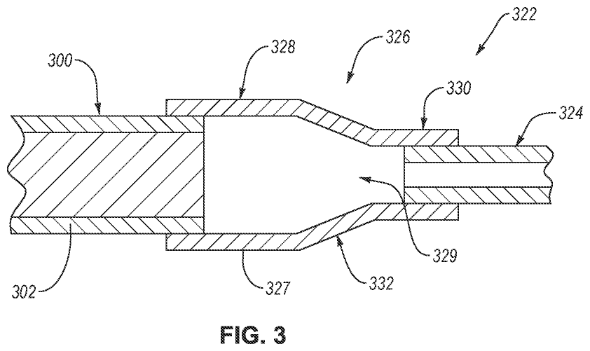

The passageway 108 may exhibit a maximum lateral dimension LD measured perpendicularly to the longitudinal axis 112. The maximum lateral dimension LD may be the diameter of the passageway 108 when the passageway 108 exhibits a generally cylindrical shape. The maximum lateral dimension LD may be selected to be about 4 mm or greater, about 5 mm or greater, about 6 mm or greater, about 7 mm or greater, about 8 mm or greater, about 9 mm or greater, about 10 mm or greater, about 12 mm or greater, about 14 mm or greater, about 16 mm or greater, about 18 mm or greater, about 20 mm or greater, about 25 mm or greater, or in ranges of about 4 mm to about 6 mm, about 5 mm to about 7 mm, about 6 mm to about 8 mm, about 7 mm to about 9 mm, about 8 mm to about 10 mm, about 9 mm to about 12 mm, about 10 mm to about 14 mm, about 12 mm to about 16 mm, about 14 mm to about 18 mm, about 16 mm to about 20 mm, or about 18 mm to about 25 mm. The maximum lateral dimension LD may be selected based on a number of factors. In an example, the maximum lateral dimension LD may be selected based on the desired flow rate of bodily fluids in the passageway 108, wherein increasing the maximum lateral dimension LD increases the flow rate (e.g., Q=A*v where Q is the flow rate, A is the cross-sectional area, and v is the velocity of the bodily fluids in the passageway 108). In an example, the maximum lateral dimension LD is selected based on the device. As used herein, the device(s) refers to the device(s) to which the conduits disclosed herein are, can be, or are configured to be attached to and may include the inlet and/or outlet of at least one of a fluid collection assembly, a fluid storage container, a vacuum source, or a hollow conduit. For instance, the maximum lateral dimension LD may be selected to be slightly smaller (e.g., the conduit 100 expands), equal to, or slightly larger than the dimension of the device if the conduit 100 forms a female attachment with the device. Also, the maximum lateral dimension LD may be selected to be smaller than the dimension of the device when the conduit 100 forms a male attachment with the device. However, it is noted that the conduit 100 may be configured to be attached to an adapter (e.g., the adaptor 326 illustrated in FIG. 3) that allows the conduit 100 to be attached to a device even if the dimensions of the conduit 100 are significantly different than the dimensions of the device.

In an embodiment, the conduit porous material 110 is formed from the same porous material that is used in the fluid collection assembly (hereafter referred to as “assembly porous material”) to which the conduit 100 is configured to be attached. Forming the conduit porous material 110 form the same material as the assembly porous material may facilitate manufacture of the conduit 100. For example, the assembly porous material is readily available and, thus, a manufacture of a fluid collection system that includes the fluid collection assembly and the conduit 100 does not need to use limited storage space to store two different porous materials. In an example, forming the conduit porous material 110 and the assembly porous material from the same material may allow the conduit porous material 110 to be at least partially formed from scraps of the assembly porous material generated during the manufacture of the fluid collection assembly, or vice versa. In an example, forming the conduit porous material 110 and the assembly porous material from the same material may allow the conduit porous material 110 and the assembly porous material to be integrally formed together, as shown in FIG. 9.

In an embodiment, as illustrated, the conduit porous material 110 extends at least substantially the entire length of the conduit 100. In other words, the conduit porous material 110 extends from or near the inlet 104 to or near the outlet 106. As such, the conduit porous material 110 provides support to substantially all of the wall 102. In an embodiment, the conduit porous material 110 only extends along a portion of the entire length of the conduit 100 which minimizes the quantity of conduit porous material 110 necessary to form the conduit 100. In such an embodiment, the conduit porous material 110 may be positioned in portions of the conduit 100 that are expected to exhibit sharp bends during use. In such example, the conduit porous material 110 may be disposed from or near the inlet 104 and extend a distance of about 15 cm to about 60 cm (e.g., 30 cm to about 45 cm) from the inlet 104 since only such portions of the conduit 100 are likely to have sharp bends formed therein. In an example, a portion of the conduit 100 is disposed in a chamber of a fluid collection assembly and the conduit porous material 110 may be omitted from at least some of the portions of the conduit 100 that are disposed in the chamber of the fluid collection assembly since the fluid collection assembly may limit bending of such portions of the conduit 100.

In an embodiment, the conduit porous material 110 does not extend outwardly from the inlet 104 and/or the outlet 106. For instance, any portions of the conduit porous material 110 that extend outwardly from the inlet 104 and/or the outlet 106 are unable to provide support to the wall 102. Further, any portions of the conduit porous material 110 that extend outwardly from the inlet 104 and/or the outlet 106 may make connecting the conduit 100 to a device difficult since such portions of the conduit porous material 110 may interfere with making connections between the inlet 104 and/or outlet 16 and a device (e.g., fluid collection assembly, another conduit, the fluid storage container, etc.) and would be required to be positioned in the device.

The wall 102 may be configured such that the conduit 100 may exhibit an average radius of curvature without kinking. The average radius of curvature that the wall 102 may exhibit without kinking may be about 0.25 cm or less, about 0.5 cm or less, about 0.75 cm or less, about 1 cm or less, about 1.25 cm or less, about 1.5 cm or less, about 1.75 cm or less, about 2 cm or less, about 2.5 cm or less, about 3 cm or less, or in ranges of about 0.25 cm to about 0.75 cm, about 0.5 cm to about 1 cm, about 0.75 cm to about 1.25 cm, about 1 cm to about 1.5 cm, about 1.25 cm to about 1.75 cm, about 1.5 cm to about 2 cm, about 1.75 cm to about 2.5 cm, or about 2 cm to about 3 cm. The average radius of curvature that the wall 102 may exhibit without kinking may depend on the thickness of the walls 102, the material that forms the wall 102, and the strength of the porous material 110.

In an embodiment, the conduit 100 may include a shape memory material (e.g., a steel, copper, or aluminum wire) disposed therein or attached thereto. The shape memory material is configured to maintain the shape of the conduit 100. For example, the shape memory material may maintain a bent shape of the conduit 100. Examples of shape memory material that may be attached to or disposed in the conduit 100 are disclosed in International Application No. PCT/US2020/042262 filed on Jul. 16, 2020 and U.S. Provisional Patent Application No. 63/094,646 filed on Oct. 21, 2020, the disclosures of each of which are incorporated herein, in its entirety, by this reference.

The conduits disclosed herein may exhibit any suitable cross-sectional shape taken along a plane that is perpendicular to the longitudinal axis of the conduits. FIGS. 2A to 2D are cross-sectional schematics of different conduits taken along a plane that is perpendicular to a longitudinal axis of the conduits, according to different embodiments. Except as otherwise disclosed herein, the conduits illustrated in FIGS. 2A to 2D are the same or substantially similar to any of the conduits disclosed herein. For example, the conduits illustrated in FIGS. 2A to 2D include at least one wall defining an inlet, an outlet downstream from the inlet, a passageway extending from the inlet to the outlet, and at least one conduit porous material disposed in at least a portion of the passageway.

Referring to FIG. 2A, the conduit 200a includes at least one wall 202a defining a passageway 208a and at least one conduit porous material 210a. The conduit porous material 210a is formed from a sheet. For example, the conduit porous materials disclosed herein are often provided in the form of a sheet.

The sheet of the conduit porous material 210a may be rolled up such that the conduit porous material 210a exhibits a generally circular cross-sectional shape. In an embodiment, as illustrated, the sheet of the conduit porous material 210a is rolled up such that opposing edges 214a of the conduit porous material 210a are positioned adjacent or proximate to each other. In such an embodiment, the conduit porous material 210a may form a central gap 216a in the center thereof. Optionally, the conduit porous material 210a may also exhibit an outer gap 218a between the opposing edges 214a of the conduit porous material 210a. When the edges 214a of the conduit porous material 210a are adjacent (e.g., contact) each other, the central gap 216a and the outer gap 218a may not be directly connected. When the edges 214 of the conduit porous material 210a are proximate (e.g., slightly spaced from) each other, the central gap 216a and the outer gap 218a may be directly connected. The central gap 216a and the outer gaps 218a increase the void space in the passageway 208a through which the bodily fluids may flow thereby increasing the flow rate of the bodily fluids through the passageway 208a. In some examples, the conduit 200a may include an additional porous material that is configured to be disposed in at least one of the central gap 216a or the outer gap 218a. The additional porous material may provide additional support to the conduit porous material 210a thereby increasing the ability of the conduit 100 to resist collapse and kinks. In an embodiment, not shown, the sheet of the conduit porous material 210a may be helically wrapped around itself thereby eliminating or minimizing the size of at least one of the central gap 216a or the outer gap 218a compared to the bending the conduit porous material 210a as shown in FIG. 2A. As such, helically wrapping the conduit porous material 210a may strengthen the conduit porous material 210a but may decrease the flow rate of the bodily fluids through the passageway 208a compared to bending the conduit porous material 210a as shown in FIG. 2A.

In some embodiments, regardless if the conduit porous material 210a is merely bent or helically wrapped, the conduit porous material 210a initially may exhibit a cross-sectional area that is smaller than the cross-sectional area of the passageway 208a, as measured along a plane that is perpendicular to a longitudinal axis of the conduit 200a. In such embodiments, the conduit porous material 210a may be disposed in a hollow conduit to form the conduit 200a. After disposing the conduit porous material 210a in the hollow conduit, the conduit porous material 210a may unbend or unwrap slightly such that the conduit porous material 210a presses against the wall 202a. In some embodiments, the conduit porous material 210a may be bend or wrapped as previously discussed herein. After bending or wrapping the conduit porous material 210a, the wall 202a may be formed around the conduit porous material 210a, for instance, by applying a fluid impermeable tape or coating to the conduit porous material 210a to form the conduit 200a.

Referring to FIG. 2B, the conduit 200b includes at least one wall 202b that defines a passageway 208b. The wall 202b and the passageway 208b may exhibit a generally circular cross-sectional shape measured along a plane that is generally perpendicular to the longitudinal axis (not shown) of the conduit 200b. The conduit 200b also includes at least one conduit porous material 210b disposed in the passageway 208b. In an example, the at least one conduit porous material 210b may exhibit a generally circular cross-sectional shape that corresponds to or is smaller than the generally circular cross-sectional shape of the passageway 208b.

Generally, devices are configured to be attached to hollow conduits exhibit a generally circular cross-sectional shape measured perpendicular to the longitudinal axis of the hollow conduits. The generally circular cross-sectional shapes of the conduit 200a illustrated in FIG. 2A and the conduit 200b illustrated in FIG. 2B may allow the conduits 200a, 200b to be directly attached (i.e., without an adaptor) to such devices, depending on the dimensions of the conduits 200a, 200b (e.g., maximum lateral dimension of the passageways thereof). However, the conduits disclosed herein may exhibit any generally non-circular cross-sectional shapes because such conduits may be attached to the devices using adaptors or because the devices are configured to be directly attached to conduits exhibiting a generally non-circular cross-sectional shape. For example, FIG. 2C illustrates a conduit 200c that exhibits a generally elongated (e.g., oblong or oval) cross-sectional shape and FIG. 2D illustrates a conduit 200d that exhibits a generally rectangular (e.g., square) cross-sectional shape. The conduits disclosed herein may also exhibit any other suitable non-circular cross-sectional shape, such as a generally hexagonal cross-sectional shape or a generally triangular cross-sectional shape.

FIG. 3 is a cross-sectional schematic of a fluid collection system 322, according to an embodiment. The fluid collection system 322 includes a conduit 300. Except as otherwise disclosed herein, the conduit 300 may include any of the conduits disclosed herein. The fluid collection system 322 also includes a device 324. As previously discussed, the device 324 may include an outlet of a fluid collection assembly, an inlet or outlet of a fluid storage container, the inlet or outlet of a vacuum source, or an inlet or outlet of a hollow conduit. The fluid collection system 322 also includes an adaptor 326. The adaptor 326 is configured to extend between and be attached to the device 324 and the conduit 300. In an embodiment, the adaptor 326 includes a conduit portion 328 that is configured to be attached to the conduit 300. The adaptor 326 may be configured to be attached to the conduit 300 using any suitable technique. In an example, the conduit portion 328 is configured to be press fitted to the conduit 300 when the conduit 300 exhibits sufficient rigidity to maintain the connection. When the conduit portion 328 is configured to be press-fitted to the conduit 300, one or more of the conduit portion 328 or the end of the conduit 300 may be tapered which may facilitate the press-fitting therebetween. Further, when the conduit portion 328 is configured to be press-fitted to the conduit 300, the conduit portion 328 may be reversibly attached to the conduit which allows the conduit 300 and the adaptor 326 to be provided separately. Providing the conduit 300 separately allows the conduit 300 to be used with a variety of devices (e.g., a device that may be directly attached to the conduit 300 or indirectly via the adaptor 326). In an example, the conduit portion 328 is permanently attached (e.g., adhesively attached or welded) to the conduit 300. The conduit portion 328 may be permanently attached to the conduit 300, for instance, when the conduit 300 does not exhibit sufficient rigidity to maintain a press-fit attachment between the conduit portion 328 and the conduit 300. Permanently attaching the conduit 300 to the conduit portion 328 only allows the conduit 300 to be indirectly attached to the device 324 and precludes the conduit 300 from being attached to a device to which the conduit 300 may be directly attached. The conduit portion 328 may be configured to form a male attachment with the conduit 300 (e.g., the conduit 300 is disposed in the conduit portion 328) or a female attachment with the conduit 300 (e.g., the conduit portion 328 is disposed in the conduit 300).

In an embodiment, the adaptor 326 is integrally formed with the wall 302 of the conduit 300 and does not include a conduit portion 328. Instead, the adaptor 326 is a portion of the wall 302 that exhibits one or more properties that are different than the rest of the wall 302. The different properties of the adaptor 326 facilitate attachment of the adaptor 326 to the device 324. For example, the different properties may include at least one of an increased rigidity (e.g., an increased thickness), a different cross-sectional shape, or different dimension(s) than the rest of the wall 302. The increased rigidity may facilitate directly press-fitting the conduit 300 to the device 324. The different cross-sectional shape and/or different dimension(s) may corresponds to (e.g., is equal to, slightly larger, or slightly smaller) the cross-sectional shape and/or dimension(s) of the device 324.

The adaptor 326 includes a device portion 330 that is configured to be attached to the device 324. Generally, the device portion 330 is configured to be reversibly attached (e.g., press-fitted) to the device 324. However, the device portion 330 may be configured to be permanently attached to the device 324. The device portion 330 may be configured to form a male attachment with the device (e.g., the device 324 is disposed in the device portion 330) or a female attachment with the device 324 (e.g., the device portion 330 is disposed in the device 324).

In some embodiments, the cross-sectional shape and/or dimension(s) of the conduit 300 are different than the cross-sectional-shape and/or dimension(s) of the device 324, respectively. In such embodiments, the adaptor 326 includes an intermediate portion 332. The intermediate portion 332 changes the cross-sectional shape and/or dimension(s) of the adaptor 326 from the conduit portion 328 to the device portion 330. For example, as illustrated, the dimension(s) of the conduit 300 may be greater than the device 324 and the intermediate portion 332 may be tapered thereby decreasing the dimension(s) of the adaptor 326 from the conduit portion 328 to the device portion 330.

The adaptor 326 includes one or more adaptor walls 327 that form the conduit portion 328, the device portion 330, and the intermediate portion 332. The adaptor walls 327 also define at least one adaptor passageway 329 that is configured to allow the bodily fluids to flow through the adaptor 326 and between the conduit 300 and the device 324. The adaptor walls 327 are formed from a fluid impermeable material to prevent the bodily fluids from leaking through the adaptor walls 327. In an example, the adaptor walls 327 may be formed from any of the fluid impermeable materials disclosed herein. In an example, the adaptor walls 327 may be formed from a rigid material to allow the adaptor 326 to be press-fitted to the conduit 300 and/or the device 324. Examples of such rigid materials include polyvinyl chloride formed without plasticizers, a metal, or another other suitable material.

It is noted that the conduit 300 may be connected to the device 324 without the adaptor 326. In an example, the conduit 300 may exhibit a size and shape that is able to connect directly to the device 324. In an example, the conduit 300 may exhibit a rigidity that allow the conduit 300 to be press-fitted to the device 324. In an example, the conduit 300 may be taped to the device 324.

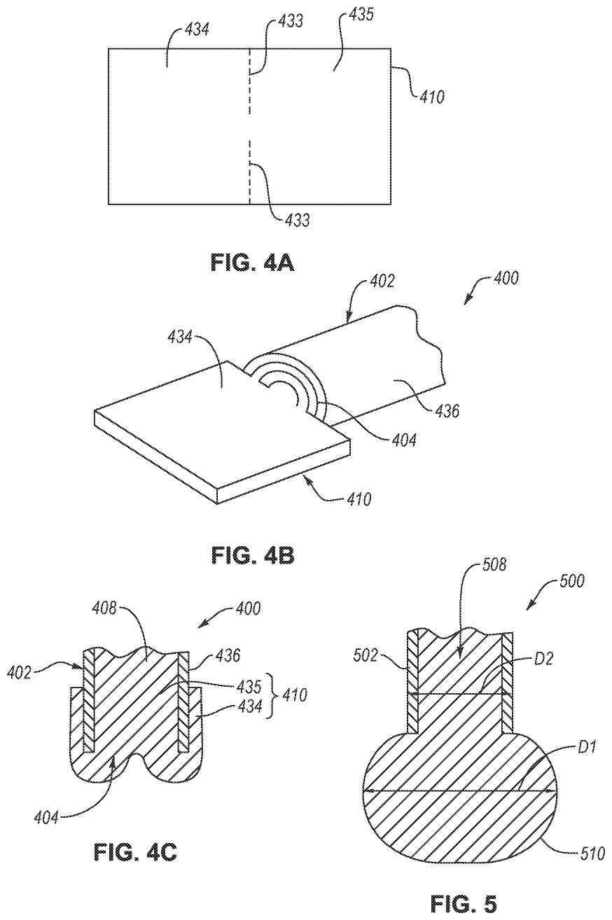

The conduits illustrated in FIGS. 1-3 are illustrated as including the conduit porous material only disposed in the passageways thereof. In other words, the conduits illustrated in FIGS. 1-3 do not include a conduit porous material extending therefrom. However, in some embodiments (as illustrated in FIGS. 4A-6B), the conduit porous material may extend outwardly from the passageways of the conduits.

FIGS. 4A and 4B illustrate a method of forming a conduit 400 that includes a conduit porous material 410 extending outwardly from the passageway 408 (illustrated in FIG. 4C), according to an embodiment. In particular, FIG. 4A is a top elevational view of the conduit porous material 410 before forming the conduit 400 and FIG. 4B is an isometric view of the conduit 400 formed using the conduit porous material 410. Except as otherwise disclosed herein, the conduit 400 may be the same or substantially similar to any of the conduits disclosed herein.

Referring to FIG. 4A, the conduit porous material 410 may be provided as a sheet, such as a generally rectangular sheet. Optionally, the conduit porous material 410 may have one or more cuts 433 formed therein. The cuts 434 are schematically illustrated using dashed lines. The cuts 433 divide the conduit porous material 410 into a first portion 434 and a second portion 435. The first portion 434 may form at least a portion of the conduit porous material 410 that extends outwardly from the passageway 408 of the conduit 400 (shown in FIG. 4C) and the second portion 435 may form at least a portion of the conduit porous material 410 that is disposed in the passageway 408 of the conduit 400.

Referring to FIG. 4B, the conduit 400 may be formed by rolling, bunching up, or otherwise collecting the second portion 435 of the conduit porous material 410. The cuts 433 allow the second portion 435 to be rolled, bunched up, or otherwise collected while the first portion 434 remains substantially undisturbed. The walls 402 may be formed around the second portion 435 or the second portion 435 may be positioned within the passageway 408 defined by one or more preformed walls 402. As such, at least a portion of the first portion 434 may extend from the walls 402 of the conduit 400. As will be discussed in more detail below, the first portion 434 may be positioned in the chamber of a fluid collection assembly which may facilitate removal of the bodily fluids from the chamber.

In an embodiment, the first portion 434 may remain in a generally sheet like configuration (e.g., the first portion 434 is not folded, rolled, bunched up, or otherwise collected). In an embodiment, the first portion 434 may be folded back onto the walls 402 of the conduit 400. For example, FIG. 4C is a cross-sectional schematic of the conduit 400 when the first portion 434 of the conduit porous material 410 is folded back onto the walls 402, according to an embodiment. The first portion 434 may be folded back such that at least a portion of the first portion 434 contacts at least a portion of the outer surface 436 of the walls 402. It has been found that the walls 402 of the conduit 400 are most likely to collapse at or near the inlet 404 of the conduit 400. However, unexpectedly, it has been found that folding the first portion 434 of the conduit porous material 410 back onto the walls 402 of the conduit 400 inhibits collapse of the walls 402 at or near the inlet 404. Further, folding the conduit porous material 410 back onto the walls 402 may decrease the quantity of assembly porous material included in the fluid collection assembly to which the conduit 400 is attached.

It is noted that FIGS. 4A-4C merely illustrate one or more methods of forming a conduit that includes a portion of a conduit porous material extending from the passageway. In an example, the conduit porous material 410 may not be cut. In an example, the conduit porous material 410 may exhibit a non-rectangular sheet-like shape, such as a shape that includes a sheet-like portion attached to a cylindrical portion.

The portion of the conduit porous material extending from the walls and passageway of the conduit may exhibit a shape other than the shape illustrated in FIGS. 4A-4C. For example, FIG. 5 is an cross-sectional schematic of a conduit 500 that includes a first portion 534 of the conduit porous material 500 extending from the passageway 508 of the conduit 500, according to an embodiment. Except as otherwise disclosed herein, the conduit 500 may be the same or substantially similar to any of the conduits disclosed herein. The first portion 534 may be configured to at least partially or substantially occupy a portion of a chamber that is not occupied by the assembly porous material. As such, the first portion 534 may exhibit a generally semi-spherical shape, a bulb-like shape, or another shape that corresponds to the portion of the chamber that is not occupied by the assembly porous material. In an example, the first portion 534 may exhibit a maximum lateral dimension D1 (e.g., diameter) that is greater than a maximum lateral dimension D2 of the walls 502 of the conduit 500 which may help the conduit portion material 510 better occupy the portions of the chamber that are not occupied by the assembly porous material. As will be discussed in more detail below, the shape of the first portion 534 may facilitate flow of the bodily fluids from the assembly porous material to the conduit porous material 510.

The conduit 500 may be formed using any suitable method. In an example, the conduit 500 may be formed using the same method as the conduit 400 illustrated in FIGS. 4A-4C except that the first portion 534 is bunched up, molded, or otherwise collected to form a shape that at least partially or substantially occupies the portion of the chamber that is not occupied by the assembly porous material. In an example, the conduit porous material 510 may be provided while exhibiting the generally semi-spherical shape, a bulb-like shape, or any other suitable shape.

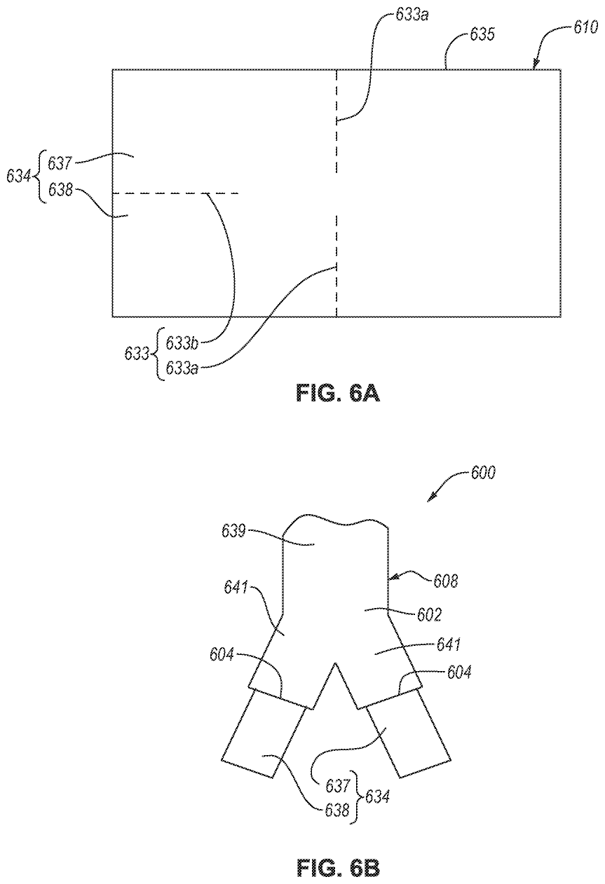

FIGS. 6A and 6B illustrate a method of forming a conduit 600 that includes a conduit porous material 610 extending outwardly from the passageway (not shown, obscured), according to an embodiment. In particular, FIG. 6A is a top elevational view of the conduit porous material 610 before forming the conduit 600 and FIG. 6B is an isometric view of the conduit 600 formed using the conduit porous material 610. Except as otherwise disclosed herein, the conduit 600 may be the same or substantially similar to any of the conduits disclosed herein.

Referring to FIG. 6A, the conduit porous material 610 may be provided as a sheet, such as a generally rectangular sheet. The conduit porous material 610 may have one or more cuts 633 formed therein. The cuts 633 are schematically illustrated using dashed lines. The conduit porous material 610 may have one or more first cuts 633 formed therein that form a first portion 634 and a second portion 635. At least a portion of the first portion 634 is configured to extend from the walls 602 of the conduit 600 and at least a portion of the second portion 635 is configured to be disposed within the passageway of the conduit 600. The conduit porous material 610 may also include one or more second cuts 633 formed therein that separate the first portion 634 into a plurality of regions (e.g., a first region 637 and a second region 638). The plurality of regions may form different and distinct flow paths through which the bodily fluids may flow through the conduit porous material 610. The plurality of regions may also allow the conduit portion material 610 to receive bodily fluids from different locations of the chamber of the fluid collection assembly. In an embodiment, the first and second cuts 633a, 633b are not parallel (e.g., perpendicular) to each other.

Referring to FIG. 6B, at least a portion of the second portion 635 may be rolled, bunched up, or otherwise collected. The rolled, bunched up, or otherwise collected portions of the second portion 635 may be disposed in a passageway defined by already formed walls 602 or the walls 602 may be formed around the such portions of the second portion 635. In an embodiment, the walls 602 may form a primary branch 639 that includes the first portion 635 disposed in the passageway defined thereby. The walls 602 may also include one or more secondary branches 641 extending from the primary branch 639. For example, the walls 602 may include secondary branches 641 for each of the regions of the first portion 634. The secondary branches 641 may define passageways that include a portion of the first portion 634 disposed therein. Each of the secondary branches 641 may define an inlet 604 and a remainder of the first portion 634 not disposed in the passageways defined by the secondary branches 641 may extend from the inlet 604. The secondary branches 641 may minimize diffusion of a vacuum pressure through all of the first portion 634 thereby concentrating the vacuum pressure at the uncovered portions of the conduit porous material 610. In an embodiment, each of the secondary branches 641 may extend the same distance from the primary branch 639. In an embodiment, at least one of the secondary branches 641 may extend a distance from the primary branch 639 that is different than at least one other secondary branch 641.

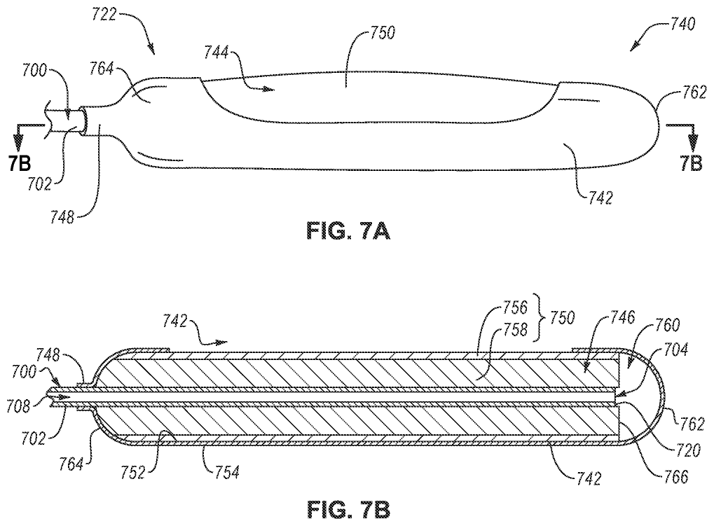

As previously discussed, the conduits disclosed herein may be used with fluid collection assemblies. FIG. 7A is an isometric view of a portion of a fluid collection system 722 that includes a fluid collection assembly 740 that is in fluid communication with a conduit 700, according to an embodiment. FIG. 7B is a cross-sectional schematic of the fluid collection system 722 taken along plane 7B-7B shown in FIG. 7A, according to an embodiment. The fluid collection assembly 740 is an example of a female fluid collection assembly for receiving and collecting bodily fluids from a female. The fluid collection assembly 740 includes a fluid impermeable barrier 742 defining at least an opening 744, a chamber 746, and a fluid outlet 748. The fluid collection assembly 740 also includes at least one assembly porous material 750 disposed in a chamber 746. The conduit 700 is disposed through the fluid outlet 748 such that an inlet 704 of the conduit 700 is disposed in the chamber 746. Except as otherwise disclosed herein, the conduit 700 may be the same or substantially similar to any of the conduits disclosed herein.

The fluid impermeable barrier 742 at least partially defines a chamber 746 (e.g., interior region) and an opening 744. For example, the interior surface(s) 752 of the fluid impermeable barrier 742 at least partially defines the chamber 746 within the fluid collection assembly 740. The fluid impermeable barrier 742 temporarily stores the bodily fluids in the chamber 746. The fluid impermeable barrier 742 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable barrier 742 substantially prevents the bodily fluids from passing through the fluid impermeable barrier 742. In an example, the fluid impermeable barrier 742 may be air permeable and fluid impermeable. In such an example, the fluid impermeable barrier 742 may be formed of a hydrophobic material that defines a plurality of pores. At least one or more portions of at least an outer surface 754 of the fluid impermeable barrier 742 may be formed from a soft and/or smooth material, thereby reducing chaffing.

In some examples, the fluid impermeable barrier 742 may be tubular (ignoring the opening), such as substantially cylindrical (as shown), oblong, prismatic, or flattened tubes. During use, the outer surface 754 of the fluid impermeable barrier 742 may contact the patient. The fluid impermeable barrier 742 may be sized and shaped to fit between the labia and/or the gluteal cleft between the legs of a female user.

The opening 744 provides an ingress route for fluids to enter the chamber 746. The opening 744 may be defined by the fluid impermeable barrier 742 such as by an inner edge of the fluid impermeable barrier 742. For example, the opening 744 is formed in and extends through the fluid impermeable barrier 742, from the outer surface 754 to the inner surface 752, thereby enabling bodily fluids to enter the chamber 746 from outside of the fluid collection assembly 740. The opening 744 may be an elongated hole in the fluid impermeable barrier 742. For example, the opening 744 may be defined as a cut-out in the fluid impermeable barrier 742. The opening 744 may be located and shaped to be positioned adjacent to a female urethra.

The fluid collection assembly 740 may be positioned proximate to the female urethral opening and the bodily fluids may enter the chamber 746 of the fluid collection assembly 740 via the opening 744. The fluid collection assembly 740 is configured to receive the bodily fluids into the chamber 746 via the opening 744. When in use, the opening 744 may have an elongated shape that extends from a first location below the urethral opening (e.g., at or near the anus or the vaginal opening) to a second location above the urethral opening (e.g., at or near the top of the vaginal opening or the pubic hair).

The opening 744 may have an elongated shape because the space between the legs of a female is relatively small when the legs of the female are closed, thereby only permitting the flow of the bodily fluids along a path that corresponds to the elongated shape of the opening 744 (e.g., longitudinally extending opening). The opening 744 in the fluid impermeable barrier 742 may exhibit a length that is measured along the longitudinal axis of the fluid collection assembly 740 that may be at least about 10% of the length of the fluid collection assembly 740, such as about 10% to about 30%, about 25% to about 40%, about 30% to about 60%, about 50% to about 75%, about 65% to about 85%, or about 75% to about 95% of the length of the fluid collection assembly 740.

The opening 744 in the fluid impermeable barrier 742 may exhibit a width that is measured transverse to the longitudinal axis of the fluid collection assembly 740 that may be at least about 10% of the circumference of the fluid collection assembly 740, such as about 10% to about 30%, about 25% to about 40%, about 30% to about 60%, about 50% to about 75%, about 65% to about 85%, or about 75% to about 100% of the circumference of the fluid collection assembly 740. The opening 744 may exhibit a width that is greater than 70% of the circumference of the fluid collection assembly 740 since the vacuum (e.g., suction) through the conduit 700 pulls the fluid through the assembly porous material 750 and into the conduit 700. In some examples, the opening 744 may be vertically oriented (e.g., having a major axis parallel to the longitudinal axis of the fluid collection assembly 740). In some examples (not shown), the opening 744 may be horizontally oriented (e.g., having a major axis perpendicular to the longitudinal axis of the fluid collection assembly 740). In an example, the fluid impermeable barrier 742 may be configured to be attached to the patient, such as adhesively attached (e.g., with a hydrogel adhesive) to the patient. According to an example, a suitable adhesive is a hydrogel layer.

In some examples, the fluid impermeable barrier 742 may define a fluid outlet 748 sized to receive the conduit 700. The at least one conduit 700 may be disposed in the chamber 746 via the fluid outlet 748. The fluid outlet 748 may be sized and shaped to form an at least substantially fluid tight seal against the conduit 700 or the at least one tube thereby substantially preventing the bodily fluids from escaping the chamber 746. It is noted that the fluid outlet 748 may be sized and shape to form an at least substantially fluid tight seal against an adaptor (e.g., adaptor 326 of FIG. 3).

The fluid impermeable barrier 742 may include markings thereon, such as one or more markings to aid a user in aligning the fluid collection assembly 740 on the patient. For example, a line on the fluid impermeable barrier 742 (e.g., opposite the opening 744) may allow a healthcare professional to align the opening 744 over the urethra of the patient. In examples, the markings may include one or more of alignment guide or an orientation indicator, such as a stripe or hashes. Such markings may be positioned to align the fluid collection assembly 740 to one or more anatomical features such as a pubic bone, etc.

The fluid collection assembly 740 includes assembly porous material 750 disposed in the chamber 746. The assembly porous material 750 may cover at least a portion (e.g., all) of the opening 744. The assembly porous material 750 may include a fluid permeable membrane 756 and a fluid permeable support 758. The assembly porous material 750 is exposed to the environment outside of the chamber 746 through the opening 744. In an embodiment, the assembly porous material 750 may be configured to wick any bodily fluids away from the opening 744, thereby preventing the bodily fluids from escaping the chamber 746. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the wicking material, such as not include adsorption of the bodily fluids into the fluid permeable support 758. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the wicking material (e.g., absorbency), such as less than about 30 wt % of the dry weight of the wicking material, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the wicking material. The wicking material may also wick the bodily fluids generally towards an interior of the chamber 746, as discussed in more detail below. In an embodiment, the assembly porous material 750 may include at least one absorbent or adsorbent material.

In an embodiment, the assembly porous material 750 may include the fluid permeable membrane 756 disposed in the chamber 746. The fluid permeable membrane 756 may cover at least a portion (e.g., all) of the opening 744. The fluid permeable membrane 756 may be composed to wick the bodily fluids away from the opening 744, thereby preventing the bodily fluids from escaping the chamber 746.

In an embodiment, the fluid permeable membrane 756 may include any material that may wick the bodily fluids. For example, the fluid permeable membrane 756 may include fabric, such as a gauze (e.g., a silk, linen, or cotton gauze), another soft fabric, another smooth fabric, or any of the other porous materials disclosed herein. Forming the fluid permeable membrane 756 from gauze, soft fabric, and/or smooth fabric may reduce chaffing caused by the fluid collection assembly 740.

The fluid collection assembly 740 may include the fluid permeable support 758 disposed in the chamber 746. The fluid permeable support 758 is configured to support the fluid permeable membrane 756 since the fluid permeable membrane 756 may be formed from a relatively foldable, flimsy, or otherwise easily deformable material. For example, the fluid permeable support 758 may be positioned such that the fluid permeable membrane 756 is disposed between the fluid permeable support 758 and the fluid impermeable barrier 742. As such, the fluid permeable support 758 may support and maintain the position of the fluid permeable membrane 756. The fluid permeable support 758 may include any material that may wick, absorb, adsorb, or otherwise allow fluid transport of the bodily fluids, such as any of the fluid permeable membrane materials disclosed herein above. For example, the fluid permeable membrane material(s) may be utilized in a more dense or rigid form than in the fluid permeable membrane 756 when used as the fluid permeable support 758. The fluid permeable support 758 may be formed from any fluid permeable material that is less deformable than the fluid permeable membrane 756. For example, the fluid permeable support 758 may include a porous polymer (e.g., nylon, polyester, polyurethane, polyethylene, polypropylene, etc.) structure or an open cell foam, such as spun nylon fiber. In some examples, the fluid permeable support 758 may be formed from a natural material, such as cotton, wool, silk, or combinations thereof. In such examples, the material may have a coating to prevent or limit absorption of fluid into the material, such as a water repellent coating. In some examples, the fluid permeable support 758 may be formed from fabric, felt, gauze, or combinations thereof.

In some examples, the fluid permeable membrane 756 may be optional. For example, the assembly porous material 750 may include only the fluid permeable support 758. In some examples, the fluid permeable support 758 may be optionally omitted from the fluid collection assembly 740. For example, the assembly porous material 750 may only include the fluid permeable membrane 756.

In an embodiment, the fluid permeable support 758 may be hydrophobic. The fluid permeable support 758 may be hydrophobic when the fluid permeable support 758 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180º. The hydrophobicity of the fluid permeable support 758 may limit absorption, adsorption, and solubility of the bodily fluids in the fluid permeable support 758 thereby decreasing the amount of bodily fluids held in the assembly porous material 750. In an embodiment, the fluid permeable membrane 756 is hydrophobic or hydrophilic. In an embodiment, the fluid permeable support 758 is more hydrophobic (e.g., exhibits a larger contact angle with water) than the fluid permeable membrane 756. The lower hydrophobicity of the fluid permeable membrane 756 may help the assembly porous material 750 receive the bodily fluids from the urethral opening while the hydrophobicity of the fluid permeable support 758 limits the bodily fluids that are retained in the assembly porous material 750.

In an embodiment, the assembly porous material 750 includes a nonwoven material instead of or in addition to at least one of the fluid permeable membrane 756 or the fluid permeable support. Examples of nonwoven materials that may be included in the assembly porous material 750 are disclosed in U.S. Provisional Patent Application No. 63/134,754 filed on Jan. 7, 2021, the disclosure of which was previously incorporated herein.

The assembly porous material 750 may at least substantially completely fill the portions of the chamber 746 that are not occupied by the conduit 700. In some examples, the assembly porous material 750 may not substantially completely fill the portions of the chamber 746 that are not occupied by the conduit 700. In such an example, the fluid collection assembly 740 includes the reservoir 760 (FIG. 1B) disposed in the chamber 746.

The reservoir 760 is a substantially unoccupied portion of the chamber 746. The reservoir 760 may be defined between the fluid impermeable barrier 742 and one or both of the fluid permeable membrane 756 and fluid permeable support 758. The bodily fluids that are in the chamber 746 may flow through the fluid permeable membrane 756 and/or fluid permeable support 758 to the reservoir 760. The reservoir 760 may retain of the bodily fluids therein.

The bodily fluids that are in the chamber 746 may flow through the fluid permeable membrane 756 and/or fluid permeable support 758 to the reservoir 760. The fluid impermeable barrier 742 may retain the bodily fluids in the reservoir 760. While depicted in the distal end region 762, the reservoir 760 may be located in any portion of the chamber 746 such as the proximal end region 764. The reservoir 760 may be located in a portion of the chamber 746 that is designed to be located in a gravimetrically low point of the fluid collection assembly when the fluid collection assembly is worn.

In some examples (not shown), the fluid collection assembly 740 may include multiple reservoirs, such as a first reservoir that is located at the portion of the chamber 746 closest to the inlet of the conduit 700 (e.g., distal end region 762) and a second reservoir that is located at the portion of the of the chamber 746 that is at or near proximal end region 764). In another example, the fluid permeable support 758 is spaced from at least a portion of the conduit 700, and the reservoir 760 may be the space between the fluid permeable support 758 and the conduit 700.

The conduit 700 may be at least partially disposed in the chamber 746. The conduit 700 may be used to remove the bodily fluids from the chamber 746. The conduit 700 at least one wall 702 defining an inlet 704, an outlet (not shown) downstream from the inlet 704, and a passageway 708. The passageway 708 may be at least partially occupied by at least one conduit porous material 710. The outlet of the conduit 700 may be operably coupled to a vacuum source, such as a vacuum pump for withdrawing fluid from the chamber 746 through the conduit 700. For example, the conduit 700 may extend into the fluid impermeable barrier 742 from the proximal end region 764 and may extend to the distal end region 762 to a point proximate to the reservoir 760 therein such that the inlet 704 is in fluid communication with the reservoir 760. The conduit 700 fluidly couples the chamber 746 with the fluid storage container (not shown) or the vacuum source (not shown).

The conduit 700 may extend through a bore in the assembly porous material 750. In an embodiment, the conduit 700 extends from the fluid outlet 748, through the bore, to a location that is proximate to the reservoir 760. In such an embodiment, the inlet 704 may not extend into the reservoir 760 and, instead, the inlet 704 may be disposed within the assembly porous material 750 (fluid permeable membrane 756 and/or fluid permeable support 758) or at a terminal end 766 thereof. For example, an end of the conduit 700 may be coextensive with or recessed within the fluid permeable membrane 756 and/or fluid permeable support 758. In an embodiment, the conduit 700 is at least partially disposed in the reservoir 760 and the inlet 704 may be extended into or be positioned in the reservoir 760. The bodily fluids collected in the fluid collection assembly 740 may be removed from the chamber 746 via the conduit 700.

Locating the inlet 704 at or near a location expected to be the gravimetrically low point of the chamber 746 when worn by a patient enables the conduit 700 to receive more of the bodily fluids than if inlet 704 was located elsewhere and reduce the likelihood of pooling (e.g., pooling of the bodily fluids may cause microbe growth and foul odors). For instance, the bodily fluids in the fluid permeable membrane 756 and the fluid permeable support 758 may flow in any direction due to capillary forces. However, the bodily fluids may exhibit a preference to flow in the direction of gravity, especially when at least a portion of the fluid permeable membrane 756 and/or the fluid permeable support 758 is saturated with the bodily fluids. Accordingly, one or more of the inlet 704 or the reservoir 760 may be located in the fluid collection assembly 740 in a position expected to be the gravimetrically low point in the fluid collection assembly 740 when worn by a patient, such as the distal end region 762.

As described in more detail below, the conduit 700 is configured to be coupled to, and at least partially extend between, one or more of the fluid storage container (not shown) and the vacuum source (not shown). In an example, the conduit 700 is configured to be directly connected to the vacuum source (not shown). In such an example, the conduit 700 may extend from the fluid impermeable barrier 742 by at least one foot, at least two feet, at least three feet, or at least six feet. In another example, the conduit 700 is configured to be indirectly connected to at least one of the fluid storage container (not shown) and the vacuum source (not shown). In some examples, the conduit is secured to a patient's skin with a catheter securement apparatus, such as a STATLOCK® catheter securement apparatus available from C. R. Bard, Inc., including but not limited to those disclosed in U.S. Pat. Nos. 6,117,163; 6,123,398; and 8,211,063, the disclosures of which are all incorporated herein by reference in their entirety.

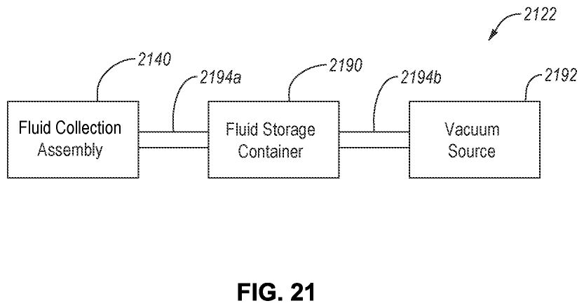

The inlet 704 and the outlet of the conduit 700 are configured to fluidly couple (e.g., directly or indirectly) the vacuum source (not shown) to the chamber 746 (e.g., the reservoir 760). As the vacuum source (FIG. 21) applies a vacuum/suction in the conduit 700, the bodily fluids in the chamber 746 (e.g., at the distal end region 762 such as in the reservoir 760) may be drawn into the inlet 704 and out of the fluid collection assembly 740 via the conduit 700. In some examples, the conduit may be frosted or opaque (e.g., black) to obscure visibility of the bodily fluids therein.

As previously discussed, the conduit 700 may be configured to be at least insertable into the chamber 746. In an example, the conduit 700 may be positioned in the chamber 746 such that a terminal end 720 of the conduit 700 is spaced from the fluid impermeable barrier 702 or other components of the fluid collection assembly 740 that may at least partially obstruct or block the inlet 704. Further, the inlet 704 of the conduit 700 may be offset relative to a terminal end 766 of the assembly porous material 750 such that the inlet 704 is closer to the proximal end region 764 of the fluid collection assembly 700 than the terminal end 766 of the assembly porous material 750. Offsetting the inlet 704 in such a manner relative to the terminal end 766 of the assembly porous material 750 allows the inlet 704 to receive bodily fluids directly from the assembly porous material 750 and, due to hydrogen bonding, pulls more bodily fluids from the assembly porous material 750.

The conduit 700 may include one or more markers (not shown) on an exterior thereof that are located to facilitate insertion of the conduit 700 into the chamber 746 and correct placement of the inlet 704 in the chamber 746. For example, the conduit 700 may include one or more markings thereon that are configured to prevent over or under insertion of the conduit 700, such as to prevent the fluid impermeable barrier 742 from obstructing or blocking the inlet 704. In another example, the conduit 700 may include one or more markings thereon that are configured to facilitate correct rotation of the conduit 700 relative to the chamber 746. The one or more markings may include a line, a dot, a sticker, or any other suitable marking.

Other embodiments of fluid impermeable barriers, fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. Pat. No. 10,390,989 filed on Sep. 8, 2016, the disclosure of each of which is incorporated herein, in its entirety, by this reference.

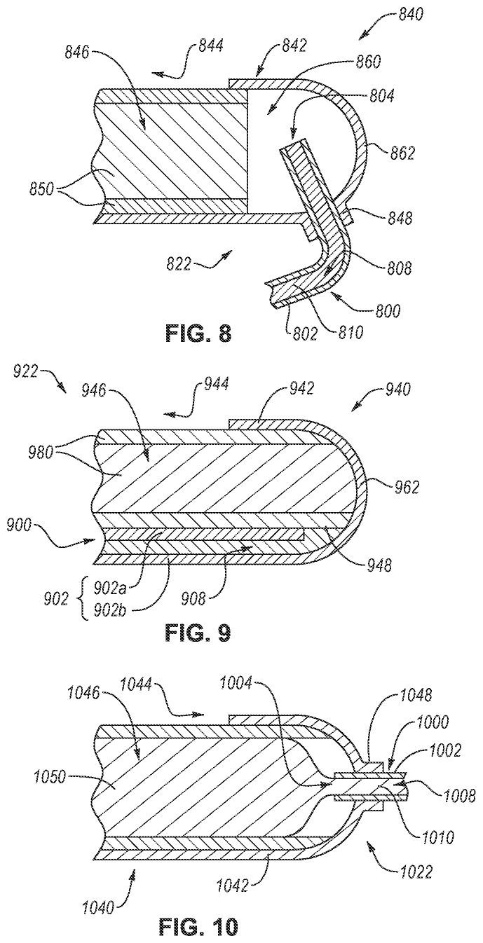

FIG. 8 is a cross-sectional schematic of a portion of a fluid collection system 822, according to an embodiment. The fluid collection system 822 includes a fluid collection assembly 840 and a conduit 800 in fluid communication with the fluid collection assembly 840. Except as otherwise disclosed herein, the conduit 800 is the same or substantially similar to any of the conduits disclosed herein. For example, the conduit 800 includes at least one wall 802 that defines an inlet 804, an outlet (not shown), and a passageway 808. The conduit 800 includes at least one conduit porous material 810 disposed in the passageway 808. Except as otherwise disclosed herein, the fluid collection assembly 840 is the same or substantially the same any the fluid collection assemblies disclosed herein. For example, the fluid collection assembly 840 includes a fluid impermeable barrier 842 that defines at least one opening 844, a chamber 846, and a fluid outlet 848. The fluid collection assembly 840 also include at least one assembly porous material 850 disposed in the chamber 846.

The fluid outlet 848 is formed at or near the distal end region 862 of the fluid collection assembly 840 instead or at or near the proximal end region (not shown). For example, the fluid outlet 848 may be formed at or near the reservoir (not shown) when the chamber 846 includes a reservoir 860 and/or at or near the expected gravimetrical low point of the fluid collection assembly 840. As such, the distance that the conduit 800 needs to extend within the chamber 846 for the inlet 804 to be at or near the reservoir 860 and/or the gravimetric low point of the chamber 846 is significantly decreased than if the fluid outlet 848 was formed at or near the proximal end region. In some examples, the assembly porous material 850 may be formed without a bore formed therein or the distance that the bore extends through the assembly porous material 850 may be decreased compared to the bore formed in the assembly porous material 850 illustrated in FIG. 7B which may increase the volume of bodily fluids that may be held within the assembly porous material 850. The conduit 800 may be disposed through the fluid outlet 848 such that the inlet 804 of the conduit 800 is adjacent to or within the assembly porous material 850.

The flexibility of the conduit 800 may allow the fluid outlet 848 to be located at or near the distal end region 862 of the fluid collection assembly 840. For example, the conduit 800 extending outwardly from the fluid outlet 848 may need to be bent at or near the fluid outlet 848 when the patient is wearing clothing or the patient is lying down. Similar bends formed in conventional hollow conduits are likely to kinky when similarly required to bend or may press into the clothing or bed.

FIG. 9 is a cross-sectional schematic of a portion of a fluid collection system 922, according to an embodiment. The fluid collection system 922 includes a fluid collection assembly 940 and a conduit 900 in fluid communication with the fluid collection assembly 940. Except as otherwise disclosed herein, the conduit 900 is the same or substantially similar to any of the conduits disclosed herein. Except as otherwise disclosed herein, the fluid collection assembly 940 is the same or substantially the same any the fluid collection assemblies disclosed herein. For example, the fluid collection assembly 940 includes a fluid impermeable barrier 942 that defines at least one opening 944, a chamber 946, and a fluid inlet 948. The fluid inlet 948 may be formed at or near the distal end region 962 (as shown) or on another portion of the fluid collection assembly 940. The fluid collection assembly 940 also include at least one assembly porous material 950 disposed in the chamber 946.

In an embodiment, at least a portion of the walls 902 of the conduit 900 are integrally formed with the fluid impermeable barrier 942 of the fluid collection assembly 940. Integrally forming the walls 902 with the fluid impermeable barrier 942 may decrease the complexity of forming the fluid collection system 922 since the fluid collection assembly 922 includes fewer parts to assembly. Integrally forming the walls 902 with the fluid impermeable barrier 942 may also make aligning and correctly positioning the conduit 900 relative to the fluid collection assembly 940 easier. In an embodiment, not shown, the conduit 900 is distinct from the fluid impermeable barrier 942 and is attached to the fluid impermeable barrier 942.

In an embodiment, the conduit 900 extends along at least a portion of the fluid impermeable barrier 942 which precludes the need to form a bore within the assembly porous material 950. In an embodiment, the fluid impermeable barrier 942 forms at least a portion (e.g., all) of the wall 902 of the conduit 900 and defines at least a portion of the passageway 908. In such an embodiment, the conduit 900 may be defined by at least one inner wall 902a and at least one outer wall 902b. At least one of the inner wall 902a or the outer wall 902b are formed by the fluid impermeable barrier 942. In an example, one of the inner or outer wall 902a, 902b are distinct from the fluid impermeable barrier 942. In such an example, the inner or outer wall 902a, 902b are is distinct from the fluid impermeable barrier 942 may be formed from at least one fluid impermeable layer that is attached to the fluid impermeable barrier 942. In an embodiment, the conduit 900 extend away from the rest of the fluid impermeable barrier 942.

In an embodiment, the porous material 950 occupies substantially all of the chamber 946 such that that chamber 946 does not define a reservoir.

FIG. 10 is a cross-sectional schematic of a portion of a fluid collection system 1022 that includes a conduit 1000 in fluid communication with a fluid collection assembly 1040, according to an embodiment. Except as otherwise disclosed herein, the conduit 1000 is the same or substantially similar to any of the conduits disclosed herein. For example, the conduit 1000 may include at least one wall 1002 that define at least an inlet 1004, an outlet (not shown), and a passageway 1008. The conduit 1000 also includes a conduit porous material 1010 disposed in the passageway 1008. Except as otherwise disclosed herein, the fluid collection assembly 1040 is the same or substantially similar to any of the fluid collection assemblies disclosed herein. For example, the fluid collection assembly 1040 may include a fluid impermeable barrier 1042 defining an opening 1044, a chamber 1046, and a fluid outlet 1048. The fluid collection assembly 1040 may also include at least one assembly porous material 1050 disposed in the chamber 1046.

The conduit porous material 1010 and at least a portion of the assembly porous material 1050 are integrally formed together (e.g., exhibit single piece construction). Integrally forming the conduit porous material 1010 and the assembly porous material 1050 together may prevent gaps from forming between the conduit porous material 1010 and the assembly porous material 1050 which may inhibit flow of the bodily fluids from the assembly porous material 1050 to the conduit porous material 1010.

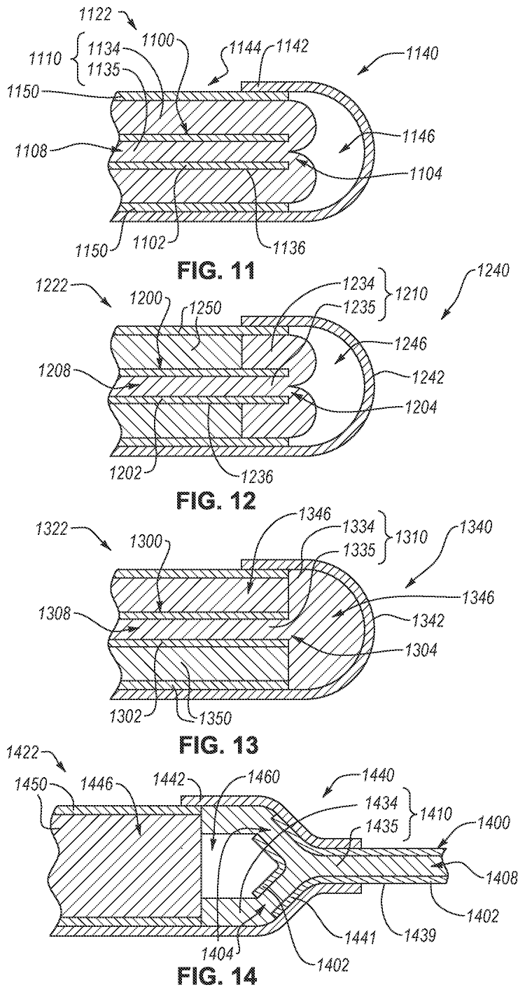

The fluid collection assemblies disclosed herein may include conduits having the conduit porous material thereof extending outwardly from the walls thereof. For example, FIG. 11 is a cross-sectional schematic of a portion of a fluid collection system 1122 that includes a conduit 1100 in fluid communication with a fluid collection assembly 1140, according to an embodiment. Except as otherwise disclosed herein, the conduit 1100 is the same or substantially similar to any of the conduits disclosed herein. For example, the conduit 1100 may include at least one wall 1102 that defines at least an inlet 1104, an outlet (not shown), and a passageway 1108. The conduit 1100 also includes a conduit porous material 1110 partially disposed in the passageway 1108. Except as otherwise disclosed herein, the fluid collection assembly 1140 is the same or substantially similar to any of the fluid collection assemblies disclosed herein. For example, the fluid collection assembly 1140 may include a fluid impermeable barrier 1142 defining a chamber 1146 and at least one assembly porous material disposed in the chamber 1146.

The conduit porous material 1110 may include a first portion 1134 extending outwardly from the passageway 1108 (e.g., from the inlet 1104) and a second portion 1135 disposed in the passageway 1108. The first portion 1134 may be folded back onto the walls 1102 of the conduit 1100. For example, the first portion 1134 may be folded back to cover all or substantially all of the outer surface 1136 of the walls 1104. When the fluid collection assembly 1140 includes the assembly porous material 1150, the first portion 1134 may support the assembly porous material 1150. The assembly porous material 1150 and the first portion 1134 of the conduit porous material 1110 may contact each other to minimize the formation of gaps therebetween.

During operation, the fluid collection assembly 1150 may receive bodily fluids into the chamber 1146 through the opening 1144. The bodily fluids may be received into the assembly porous material 1150 and flow into the first portion 1134 of the conduit porous material 1110. The bodily fluids may then flow into the second portion 1135 from the first portion 1134 where the vacuum pressure applied to the conduit 1100 suctions the bodily fluids through the conduit 1100. The hydrogen bonding between the water molecules of the bodily fluids causes the bodily fluids that are being suctioned through the second portion 1135 of the conduit porous material 1110 to pull additional bodily fluids from the first portion 1134 into the second portion 1135. Further, the hydrogen bonding causes the bodily fluids that are being pulled into the second portion 1135 to pull the bodily fluids into the first portion 1134 of the conduit porous material 1110 from the assembly porous material 1150. It is noted that any gaps between the assembly porous material 1150 and the first portion 1134 may break the hydrogen bonding between the water molecules in the assembly porous material 1150 and the first portion 1134. The lack of hydrogen bonding may prevent the pulling of the bodily fluids from the assembly porous material 1150 into the conduit porous material 1110. Instead, the bodily fluids flowing from the assembly porous material 1150 to the conduit porous material 1110 may, rely on wicking and gravity which may be slower. The hydrogen bonding between the water molecules and the direct contact between the conduit porous material 1110 and the assembly porous material 1150 decreases the need to position the inlet 1104 of the conduit 1100 at or near the expected gravimetric low point of the chamber 1146. The first portion 1134 may also facilitate alignment of the conduit 1100 in the chamber 1146.

FIG. 12 is a cross-sectional schematic of a portion of a fluid collection system 1222 that includes a conduit 1200 in fluid communication with a fluid collection assembly 1240, according to an embodiment. Except as otherwise disclosed herein, the conduit 1200 is the same or substantially similar to any of the conduits disclosed herein. For example, the conduit 1200 may include at least one wall 1202 that defines at least an inlet 1204, an outlet (not shown), and a passageway 1208. The conduit 1200 also includes a conduit porous material 1210 partially disposed in the passageway 1208. Except as otherwise disclosed herein, the fluid collection assembly 1240 is the same or substantially similar to any of the fluid collection assemblies disclosed herein. For example, the fluid collection assembly 1240 may include a fluid impermeable barrier 1242 and at least one assembly porous material 1250.

The conduit porous material 1210 may include a first portion 1234 extending outwardly from the passageway 1208 and a second portion 1235 disposed in the passageway 1208. The first portion 1234 may be folded back onto the walls 1202 of the conduit 1200. For example, the first portion 1234 may be folded back to cover only a portion of the outer surface 1236 of the walls 1202. The assembly porous material 1250 may define a cutout or be otherwise configured to accommodate and abut the first portion of 1234 to allow bodily fluids to be pulled from the assembly porous material 1250 to the first portion 1234 responsive to the bodily fluids being pulled from the first portion 1234 into the second portion 1235. The hydrogen bonding between the water molecules and the direct contact between the conduit porous material 1210 and the assembly porous material 1250 decreases the need to position the inlet 1204 of the conduit 1200 at or near the expected gravimetric low point of the chamber 1246. The first portion 1234 may also facilitate alignment of the conduit 1200 in the chamber 1246.

FIG. 13 is a cross-sectional schematic of a portion of a fluid collection system 1322 that includes a conduit 1300 in fluid communication with a fluid collection assembly 1340, according to an embodiment. Except as otherwise disclosed herein, the conduit 1300 is the same or substantially similar to any of the conduits disclosed herein. For example, the conduit 1300 may include at least one wall 1302 that defines at least an inlet 1304, an outlet (not shown), and a passageway 1308. The conduit 1300 also includes a conduit porous material 1310 partially disposed in the passageway 1308. Except as otherwise disclosed herein, the fluid collection assembly 1340 is the same or substantially similar to any of the fluid collection assemblies disclosed herein. For example, the fluid collection assembly 1340 may include a fluid impermeable barrier 1342 defining a chamber 1346 and at least one assembly porous material 1350 disposed in the chamber 1346.

The conduit porous material 1310 includes a first portion 1334 extending from the inlet 1304 and a second portion 1335 disposed in the passageway 1308. The first portion 1334 exhibits a shape that is configured to at least partially or substantially completely occupy a portion of the chamber 1346 that is not occupied by the assembly porous material 1350 and the rest of the conduit 1300. In other words, the first portion 1334 may prevent the chamber 1346 from forming a reservoir or may inhibit the volume of the reservoir.

The first portion 1334 may facilitate the flow of the bodily fluids from the assembly porous material 1350 and into the passageway 1308 of the conduit 1300. For example, as previously discussed, the hydrogen bonding between water molecules may pull the bodily fluids from the assembly porous material 1350, into the first portion 1334 of the conduit porous material 1350, and into the second portion 1335 of the conduit porous material 1350. When the chamber 1346 defines a reservoir, at least some of the bodily fluids may enter the reservoir. The bodily fluids may not be removed from the reservoir until the quantity of bodily fluids in the reservoir is sufficient for the bodily fluids to reach the inlet 1304 of the conduit 1300. As such, the presence of the reservoir may inhibit removal of substantially all of the bodily fluids from the chamber 1346 in a quick manner though, it is noted, the reservoir may increase the quantity of bodily fluids stored in the chamber 1346. Further, at least substantially occupying the portions of the chamber 1346 that do not include the assembly porous material 1350 with the conduit 1300 inhibits twisting and/or collapse of such portions of the chamber 1346 during use of the fluid collection assembly 1340. The hydrogen bonding between the water molecules, the direct contact between the conduit porous material 1310 and the assembly porous material 1350, and the decreased volume of any unoccupied portions of the chamber 1346 decreases the need to position the inlet 1304 of the conduit 1300 at or near the expected gravimetric low point of the chamber 1346. The first portion 1334 may also facilitate alignment of the conduit 1300 in the chamber 1346.

FIG. 14 is a cross-sectional schematic of a portion of a fluid collection system 1422 that includes a conduit 1400 in fluid communication with a fluid collection assembly 1440, according to an embodiment. Except as otherwise disclosed herein, the conduit 1400 is the same or substantially similar to any of the conduits disclosed herein. For example, the conduit 1400 may include at least one wall 1402 that defines at least an inlet 1404, an outlet (not shown), and a passageway 1408. The conduit 1400 also includes a conduit porous material 1410 partially disposed in the passageway 1408. Except as otherwise disclosed herein, the fluid collection assembly 1440 is the same or substantially similar to any of the fluid collection assemblies disclosed herein. For example, the fluid collection assembly 1440 may include a fluid impermeable barrier 1442 defining a chamber 1446 and at least one assembly porous material 1450 disposed in the chamber 1446.

The conduit 1400 is substantially similar to the conduit 600 illustrated in FIG. 6B. For example, the first portion 1434 of the conduit porous material 1410 may include a plurality of distinct regions that form different paths through which the bodily fluids may flow. The walls 1402 may also include a primary branch 1439 and one or more secondary branches 1441 extending therefrom. The plurality of regions of the first portion 1434 allows the conduit 1400 to remove bodily fluids from a plurality of different regions of the chamber 1446. It also may inhibit pooling of the bodily fluids in unoccupied portions of the chamber 1446. The hydrogen bonding between the water molecules and the direct contact between the conduit porous material 1410 and the assembly porous material 1450 decreases the need to position the inlet 1404 of the conduit 1400 at or near the expected gravimetric low point of the chamber 1446. The plurality of regions may also facilitate alignment of the conduit 1400 in the chamber 1446.

In an embodiment, as illustrated, the chamber 1446 includes a reservoir 1460. The reservoir 1460 may include the substantially unoccupied between the plurality of regions of the first portion 1434 of the conduit porous material 1410. However, the plurality of regions facilitates the removal of the bodily fluids from the reservoir 1460 since the plurality of regions of the first portion 1434 inhibits pooling. In an embodiment, the assembly porous material 1450 at least partially occupies the space between the plurality of regions of the first portion 1434 to further inhibit pooling of the bodily fluids in such unoccupied space.

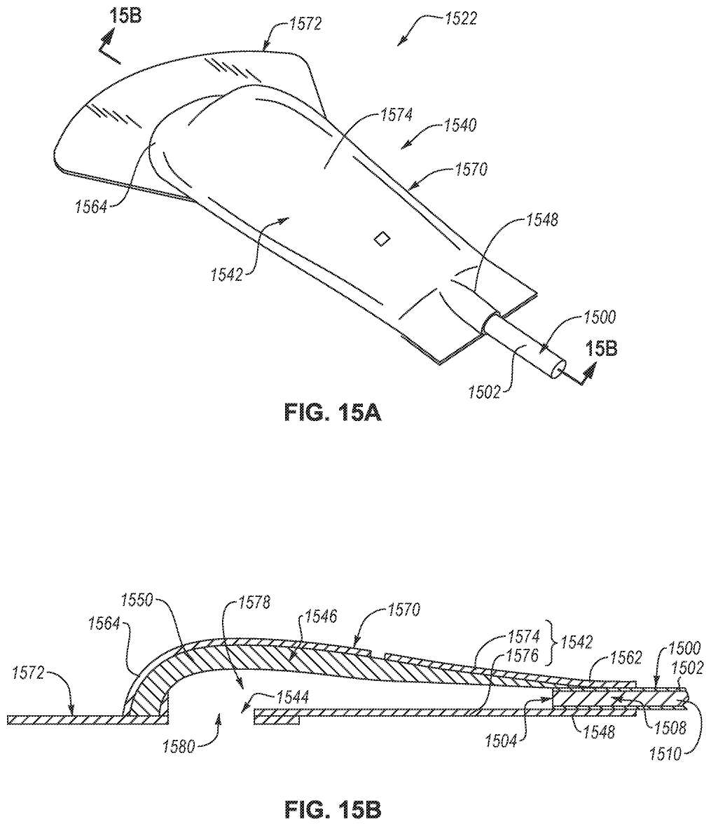

The conduits disclosed herein may be included in fluid collection systems that include a male fluid collection assembly that is configured to receive bodily fluids from a male (e.g., from a penis). For example, FIG. 15A is an isometric view of a portion of a fluid collection system 1522 that includes a conduit 1500 in fluid communication with a fluid collection assembly 1540, according to an embodiment. FIG. 15B is a cross-sectional schematic of the fluid collection assembly 1540 taken along plane 15B-15B shown in FIG. 15A, according to an embodiment. The fluid collection assembly 1540 is an example of a male fluid collection assembly though, in some embodiments, the fluid collection assembly 1540 may be used to receive bodily fluids from a female urethral opening. Except as otherwise disclosed herein, the conduit 1500 is the same or substantially similar to any of the conduits disclosed herein. For example, the conduit 1500 may include at least one wall 1502 that define at least an inlet 1504, an outlet (not shown), and a passageway 1508. The conduit 1500 also includes a conduit porous material 1510 disposed in the passageway 1508. Except as otherwise disclosed herein, the fluid collection assembly 1540 is the same or substantially similar to any of the fluid collection assemblies disclosed herein. For example, the fluid collection assembly 1540 may include a fluid impermeable barrier 1542 defining an opening 1544, a chamber 1546, and a fluid outlet 1548. The fluid collection assembly 1540 may also include at least one assembly porous material 1550 disposed in the chamber 1546.

The fluid collection assembly 1540 includes a sheath 1570 and a base 1572. The base 1572 is configured to be attached (e.g., permanently attached to or configured to be permanently attached) to the sheath 1570. The base 1572 is also configured to be attached to the region about the urethral opening (e.g., penis) of the patient.

The sheath 1570 includes the fluid impermeable barrier 1542 that is at least partially formed from a first panel 1574 and a second panel 1576. The first panel 1574 and the second panel 1576 may be attached or integrally formed together (e.g., exhibits single piece construction). In an embodiment, as illustrated, the first panel 1574 and the second panel 1576 are distinct sheets. The fluid impermeable barrier 1542 also defines a chamber 1546 between the first panel 1574 and the second panel 1576, an opening 1544 at a proximal end region 1564 of the sheath 1570, and an fluid outlet 1548 at a distal end region 1562 of the sheath 1570. The sheath 1570 also includes at least one assembly porous material 1550 disposed in the chamber 1546.

The inner surface(s) of the fluid impermeable barrier 1542 (e.g., inner surfaces of the first and second panels 1574, 1576 at least partially defines the chamber 1546 within the fluid collection assembly 1540. The fluid impermeable barrier 1542 temporarily stores the bodily fluids in the chamber 1546. The fluid impermeable barrier 1542 may be formed from any of the fluid impermeable materials disclosed herein. As such, the fluid impermeable barrier 1542 substantially prevents the bodily fluids from passing through the fluid impermeable barrier 1542.

In an embodiment, at least one of the first panel 1574 or the second panel 1576 is formed from an at least partially transparent fluid impermeable material, such as polyethylene, polypropylene, polycarbonate, or polyvinyl chloride. Forming at least one of the first panel 1574 or the second panel 1576 from an at least partially transparent fluid impermeable material allows a person (e.g., medical practitioner) to examiner the penis. In some embodiments, both the first panel 1574 and the second panel 1576 are formed from at least partially transparent fluid impermeable material. Selecting at least one of the first panel 1574 or the second panel 1576 to be formed from an at least partially transparent impermeable material allows the penis to be examined without detaching the entire fluid collection assembly 1540 from the region about the penis. For example, the chamber 1546 may include a penis receiving area 1578 that is configured to receive the penis of the individual when the penis extends into the chamber 1546. The penis receiving area 1578 may be defined by at least the assembly porous material 1550 and at least a portion of the at least partially transparent material of the first panel 1574 and/or the second panel 1576. In other words, the assembly porous material 1550 is positioned in the chamber 1546 such that the assembly porous material 1550 is not positioned between the penis and at least a portion of the transparent portion of the first panel 1574 and/or second panel 1576 when the penis is inserted into the chamber 1546 through the opening 1544. The assembly porous material 1550 is generally not transparent and, thus, the portion of the at least partially transparent material of the first panel 1574 and/or the second panel 1576 that defines the penis receiving area 1578 forms a window which allows the person to view into the penis receiving area 1578 and examine the penis.

The opening 1544 defined by the fluid impermeable barrier 1542 provides an ingress route for fluids to enter the chamber 1546 when the penis is a buried penis and allow the penis to enter the chamber 1546 (e.g., the penis receiving area 1578) when the penis is not buried. The opening 1544 may be defined by the fluid impermeable barrier 1542 (e.g., an inner edge of the fluid impermeable barrier 1542). For example, the opening 1544 is formed in and extends through the fluid impermeable barrier 1542 thereby enabling bodily fluids to enter the chamber 1546 from outside of the fluid collection assembly 1540.

The fluid impermeable barrier 1542 defines the fluid outlet 1548 sized to receive the conduit 1500. The conduit 1500 may be at least partially disposed in the chamber 1546 or otherwise in fluid communication with the chamber 1546 through the fluid outlet 1548. The fluid outlet 1548 may be sized and shaped to form an at least substantially fluid tight seal against the conduit 1500 thereby substantially preventing the bodily fluids from escaping the chamber 1546. In an embodiment, the fluid outlet 1548 may be formed from a portion of the first panel 1574 and the second panel 1576 that are not attached or integrally formed together. In such an embodiment, the fluid impermeable barrier 1542 may not include a cap exhibiting a rigidity that is greater than the portions of the fluid impermeable barrier 1542 thereabout which may facilitate manufacturing of the fluid collection assembly 1540 may decreasing the number of parts that are used to form the fluid collection assembly 1540 and may decrease the time required to manufacture the fluid collection assembly 1540. The lack of the cap may make securing the conduit 1500 to the fluid outlet 1548 using interference fit to be difficult though, it is noted, attaching the conduit 1500 to the fluid outlet 1548 may still be possible. As such, the conduit 1500 may be attached to the fluid outlet 1548 (e.g., to the first and second panels 1574, 1576) using an adhesive, a weld, or otherwise bonding the fluid outlet 1548 to the fluid outlet 1548. Attaching the conduit 1500 to the fluid outlet 1548 may prevent leaks and may prevent the conduit 1500 from inadvertently becoming detached from the fluid outlet 1548. In an example, the conduit 1500 may be attached to the fluid outlet 1548 in the same manufacturing step that attaches the first and second panels 1574, 1576 together.

As previously discussed, the sheath 1570 includes at least one assembly porous material 1550 disclosed in the chamber 1546. The assembly porous material 1550 may direct the bodily fluids to one or more selected regions of the chamber 1546, such as away from the penis and towards the fluid outlet 1548. The assembly porous material 1550 may be formed from any of the porous materials disclosed herein. In an example, the assembly porous material 1550 may be formed from a single layer, two layers (e.g., a fluid permeable membrane extending across the opening 1544 and a fluid permeable support since the fluid permeable membrane may be formed from a relatively foldable, flimsy, or otherwise easily deformable material), or three or more layers. In an example, the assembly porous material 1550 may be formed from a nonwoven material or a woven material (e.g., spun nylon fibers). In an example, the assembly porous material 1550 may include at least one material exhibiting substantially no absorption or at least one absorbent or adsorbent material.

In an embodiment, the assembly porous material 1550 may be a sheet. Forming the assembly porous material 1550 as a sheet may facilitate the manufacturing of the fluid collection assembly 1540. For example, forming the assembly porous material 1550 as a sheet allows the first panel 1574, the second panel 1576, and the assembly porous material 1550 to each be sheets. During the manufacturing of the fluid collection assembly 1540, the first panel 1574, the second panel 1576, and the assembly porous material 1550 may be stacked and then attached to each other in the same manufacturing step. For instance, the assembly porous material 1550 may exhibit a shape that is the same size or, more preferably, slightly smaller than the size of the first panel 1574 and the second panel 1576. As such, attaching the first panel 1574 and the second panel 1576 together along the outer edges thereof may also attach the assembly porous material 1550 to the first panel 1574 and the second panel 1576. The assembly porous material 1550 may be slightly smaller than the first panel 1574 and the second panel 1576 such that the first panel 1574 and/or the second panel 1576 extend around the assembly porous material 1550 such that the assembly porous material 1550 does not form a passageway through the fluid impermeable barrier 1542 through which the bodily fluids may leak. Also, attaching the assembly porous material 1550 to the first panel 1574 and/or the second panel 1576 may prevent the assembly porous material 1550 from significantly moving in the chamber 1546, such as preventing the assembly porous material 1550 from bunching together near the fluid outlet 1548. In an example, the assembly porous material 1550 may be attached to the first panel 1574 or the second panel 1576 (e.g., via an adhesive) before or after attaching the first panel 1574 to the second panel 1576. In an example, the assembly porous material 1550 may merely be disposed in the chamber 1546 without attaching the assembly porous material 1550 to at least one of the first panel 1574 or the second panel 1576. In an embodiment, the assembly porous material 1550 may exhibit shapes other than a sheet, such as a hollow generally cylindrical shape.

Generally, the sheath 1570 is substantially flat when the penis is not in the penis receiving area 1578 and the sheath 1570 is resting on a flat surface. The sheath 1570 is substantially flat because the fluid impermeable barrier 1542 is formed from the first panel 1574 and the second panel 1576 instead of a generally tubular fluid impermeable barrier. Further, as previously discussed, the assembly porous material 1550 may be a sheet, which also causes the sheath 1570 to be substantially flat. The sheath 1570 may also be substantially flat because the fluid collection assembly 1540 may not include relatively rigid rings or caps that exhibit a rigidity that is greater than the portions of the fluid impermeable barrier 1542 thereabout since such rings and caps may inhibit the sheath 1570 being substantially flat. It is noted that the sheath 1570 is described as being substantially flat because at least one of the assembly porous material 1550 may cause a slight bulge to form in the sheath 1570 depending on the thickness of the assembly porous material 1550, the fluid outlet 1548 and/or conduit 1500 may cause a bulge thereabout, or the base 1572 may pull on portions of the sheath 1570 thereabout. It is also noted that the sheath 1570 may also be compliant and, as such, the sheath 1570 may not be substantially flat during use since, during use, the sheath 1570 may rest on a non-flat surface (e.g., may rest on the testicles, the perineum, and/or between the thighs) and the sheath 1570 may conform to the surface of these shapes.

The ability of the sheath 1570 to be substantially flat when the penis is not in the penis receiving area 1578 and the sheath 1570 is resting on a flat surface allows the fluid collection assembly 1540 to be used with a buried and a non-buried penis. For example, when the fluid collection assembly 1540 is being used with a buried penis, the penis does not extend into the penis receiving area 1578 which causes the sheath 1570 to lie relatively flat across the aperture 1580 of the base 1572. When the sheath 1570 lies relatively flat across the aperture 1580, the assembly porous material 1550 extends across the opening 1544 and the aperture 1580 and is in close proximity to the buried penis. As such, the assembly porous material 1550 prevents or inhibits pooling of bodily fluids discharged from the buried penis against the skin of the individual since the assembly porous material 1550 will receive and remove at least a significant portion of the bodily fluids that would otherwise pool against the skin of the individual. Thus, the skin of the individual remains dry thereby improving comfort of using the fluid collection assembly 1540 and preventing skin degradation. However, unlike other conventional fluid collection assemblies that are configured to be used with buried penises, the fluid collection assembly 1540 may still be used with a non-buried penis since the non-buried penis can still be received into the penis receiving area 1578, even when the penis is fully erect. Additionally, the ability of the sheath 1570 to be substantially flat allows the fluid collection assembly 1540 to be used more discretely than if the sheath 1570 was not substantially flat thereby avoiding possibly embarrassing scenarios.

When the sheath 1570 is substantially flat, the assembly porous material 1550 occupies substantially all of the chamber 1546 and the penis receiving area 1578 is collapsed (shown as being non-collapsed in FIG. 15B for illustrative purposes to show the penis receiving area 1578). In other words, the sheath 1570 may not define a region that is constantly unoccupied by the assembly porous material 1550. When the assembly porous material 1550 occupies substantially all of the chamber 1546, the bodily fluids discharged into the chamber 1546 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 1574, the second panel 1576, and the assembly porous material 1550 may be selected to be relatively flexible. The first panel 1574, the second panel 1576, and the assembly porous material 1550 are relatively flexible when the first panel 1574, the second panel 1576, and the assembly porous material 1550, respectively, are unable to maintain their shape when unsupported. The flexibility of the first panel 1574, the second panel 1576, and the assembly porous material 1550 may allow the sheath 1570 to be substantially flat, as discussed above. The flexibility of the first panel 1574, the second panel 1576, and the assembly porous material 1550 may also allow the sheath 1570 to conform to the shape of the penis even when the size and shape of the penis changes (e.g., becomes erect) and to minimize any unoccupied spaces in the chamber 1546 in which bodily fluids may pool.

As previously discussed, the fluid collection assembly 1540 includes a base 1572 that is configured to be attached to the sheath 1570. For example, the base 1572 is configured to be permanently attached to the sheath 1570. The base 1572 is configured to be permanently attached to the sheath 1570 when, for example, when the fluid collection assembly 1540 is provided with the base 1572 permanently attached to the sheath 1570 or the base 1572 is provided without being permanently attached to the sheath 1570 but is configured to be permanently attached to the sheath 1570 at some point in the future. Permanently attached means that the sheath 1570 cannot be detached from the base 1572 without damaging at least one of the sheath 1570 or the base 1572, using a blade to separate the sheath 1570 from the base 1572, and/or using chemicals to dissolve the adhesive that attaches the sheath 1570 from the base 1572. The base 1572 may be permanently attached to the sheath 1570 using an adhesive, sewing, heat sealing, RF welding, or US welding. In an embodiment, the base 1572 is configured to be reversibly attached to the sheath 1570. In an embodiment, the base 1572 is integrally formed with the sheath 1570.

As previously discussed, the base 1572 includes an aperture 1580. The base 1572 is permanently attached to the first end region 120 of the sheath 1570 such that the aperture 1580 is aligned with the opening 1544.

The base 1572 is sized, shaped, and made of a material to be coupled to the skin that surrounds the penis (e.g., mons pubis, thighs, testicles, and/or perineum) and have the penis disposed therethrough. For example, the base 1572 may define an aperture 1580 configured to have the penis positioned therethrough. In an example, the base 1572 may exhibit the general shape or contours of the skin surface that the base 1572 is configured to be coupled with. The base 1572 may be flexible, thereby allowing the base 1572 to conform to any shape of the skin surface and mitigate the base 1572 pulling the on skin surface. The base 1572 may extend laterally past the sheath 1570 thereby increasing the surface area of the skin of the individual to which the fluid collection assembly 1540 may be attached compared to a substantially similar fluid collection assembly 1540 that did not include a base.

As previously discussed, the fluid collection assembly 1540 includes the conduit 1500. The conduit 1500 may be the same or substantially similar to any of the conduits disclosed herein. For example, the conduit 1500 includes a conduit porous material 1510 disposed in the passageway 1508. The conduit porous material 1510 may be distinct from the assembly porous material 1550 (as shown) or may be integrally formed with the assembly porous material 1550 (as shown in FIGS. 10 and 17).

The inlet 1504 of the conduit 1500 may be located near the distal end region 1562 of the sheath 1570 which is expected to be the gravimetrically low point of the chamber 1546 when worn by a patient. Locating the inlet 1504 at or near the distal end region 1562 of the sheath 1570 enables the conduit 1500 to receive more of the bodily fluids than if the inlet of the conduit 1500 was located elsewhere and reduce the likelihood of pooling (e.g., polling of the bodily fluids may cause microbe growth and foul odors).

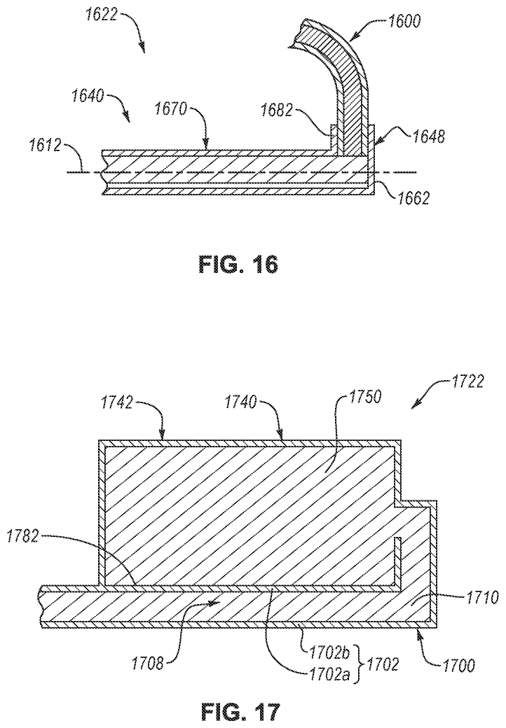

The conduit 150 is illustrated as extending from the distal end region 1562 of the sheath 1570 in a direction that is generally parallel to a longitudinal axis of the sheath 1570 when the sheath 1570 is lying on a flat surface. However, due to the flexibility of the conduit 1500, the conduit 1500 may extend from the distal end region 1562 at an angle that is not generally parallel to the longitudinal axis of the sheath 1570. For example, FIG. 16 is a cross-sectional schematic of a portion of a fluid collection system 1622 that includes a conduit 1600 in fluid communication with a fluid collection assembly 1640, according to an embodiment. Except as otherwise disclosed herein, the conduit 1600 and the fluid collection assembly 1640 are the same or substantially similar to any of the conduits and fluid collection assemblies disclosed herein, respectively. The fluid collection assembly 1640 includes a sheath 1670 defining a fluid outlet 1648 at or near the distal end region 1662 of the sheath 1670. The fluid outlet 1648 is configured to allow the conduit 1600 to extend from the sheath 1670 at a direction that is not parallel to (e.g., perpendicular or oblique) relative to a longitudinal axis 1612 of the sheath 1670 when the sheath 1670 is lying on a flat surface. For example, the fluid outlet 1648 may include at least one wall 1682 that are configured to abut the conduit 1600. The wall 1682 may extend from the rest of the sheath 1670 in a direction that is not parallel to the longitudinal axis 1612 of the sheath 1670 thereby causing the conduit 1600 to extend from the sheath 1670 in a similar direction. The conduit 1600 may extend from the sheath 1670 in a direction that is not parallel to the longitudinal axis of the sheath 1670 since the conduit 1600 is able to be bent for the reasons previously disclosed herein.

The fluid impermeable barriers of the male fluid collection assemblies disclosed herein may form at least a portion of the wall (e.g., define at least a portion of the passageway) of the conduit attached thereto. For example, FIG. 17 is a cross-sectional schematic of a portion of a fluid collection system 1722 that includes a conduit 1700 in fluid communication with a fluid collection assembly 1740, according to an embodiment. Except as otherwise disclosed herein, the conduit 1700 and the fluid collection assembly 1740 are the same as or substantially similar to any of the conduits and fluid collection assemblies, respectively, disclosed herein. It is noted that FIGS. 15B to 16 are side cross-sectional schematics of at least a portion of a fluid collection system whereas FIG. 17 is a top cross-sectional schematic of a portion of the fluid collection system 1722.

Similar to the fluid collection system 922 illustrated in FIG. 9, the conduit 1700 extends along at least a portion of the fluid impermeable barrier 1742. The conduit 1700 may extend along a lateral side 1784 of the fluid collection assembly 1740 to prevent the weight of the conduit 1700 from resting on the penis of the patient which may increase patient discomfort. In an embodiment, not shown, the conduit 1700 is distinct from the fluid impermeable barrier 842 and is attached to the fluid impermeable barrier 1742. In an embodiment, the fluid impermeable barrier 1742 forms at least a portion (e.g., all) of the wall 1702 of the conduit 1700 and defines at least a portion of the passageway 1708. In such an embodiment, the conduit 1700 may be defined by at least one inner wall 1702a and at least one outer wall 1702b. In an example, one of the inner or outer wall 1702a, 1702b are distinct from the fluid impermeable barrier 1742. In such an example, the inner or outer wall 1702a, 1702b that is distinct from the fluid impermeable barrier 1742 may be formed from at least one fluid impermeable layer that is attached to the fluid impermeable barrier 1742.

Similar to the fluid collection system 1022 illustrated in FIG. 10, the conduit porous material 1710 and at least a portion of the assembly porous material 1750 are integrally formed together (e.g., exhibit single piece construction). For example, the conduit porous material 1710 and at least a portion of the assembly porous material 1750 may be formed from the same sheet with a cut or portion removed therefrom to allow for a portion of the walls 1702 and/or fluid impermeable barrier 1742 to be formed between a portion of the conduit porous material 1710 and the assembly porous material 1750.

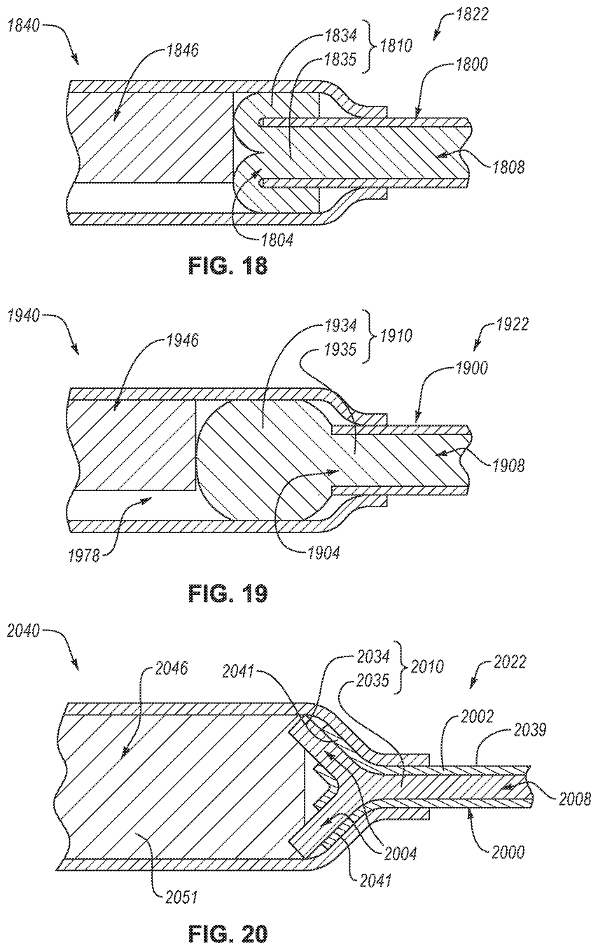

The male fluid collection assemblies disclosed herein may be used with conduits including a porous of the conduit porous material extending from the inlets thereof. For example, FIG. 18 is a cross-sectional schematic of a portion of a fluid collection system 1822 that includes a conduit 1800 in fluid communication with a fluid collection assembly 1840, according to an embodiment. Except as otherwise disclosed herein, the conduit 1800 and the fluid collection assembly 1840 are the same or substantially similar to any of the conduits and fluid collection assemblies disclosed herein, respectively. The conduit 1800 includes a conduit porous material 1810 including a first portion 1834 and a second portion 1835. At least a portion of the first portion 1834 extends outwardly from the passageway 1808 (e.g., from the inlet 1804) and at least a portion of the second portion 1835 is disposed in the passageway 1808. In an embodiment, the first portion 1834 may be folded back which, as previously discussed, may inhibit collapse of the conduit 1800 at or near the inlet 1804. The conduit porous material 1810 may abut the assembly porous material 1850 to facilitate pulling the bodily fluids from the assembly porous material 1850 to the conduit porous material 1810 via hydrogen bonding. The conduit porous material 1810 may inhibit twisting and/or collapse of the chamber 1846, facilitate alignment of the conduit 1800, and, as previously discussed, may decrease the need to have the inlet 1804 of the conduit 1800 positioned at or near the expected gravimetric low point of the chamber 1846.

FIG. 19 is a cross-sectional schematic of a portion of a fluid collection system 1922 that includes a conduit 1900 in fluid communication with a fluid collection assembly 1940, according to an embodiment. Except as otherwise disclosed herein, the conduit 1900 and the fluid collection assembly 1940 are the same or substantially similar to any of the conduits and fluid collection assemblies disclosed herein, respectively. The conduit 1900 includes a conduit porous material 1910 including a first portion 1934 and a second portion 1935. At least a portion of the first portion 1934 extends outwardly from the passageway 1908 and at least a portion of the second portion 1935 is disposed in the passageway 1908. The first portion 1934 may be configured to substantially occupy portions of the chamber 1946 near the inlet 1904 of the conduit 1900. For example, the first portion 1934 may be configure to exhibit substantially occupy the portions of the chamber 1946 that are not occupied by the assembly porous material 1950 or form part of the penis receiving area 1978. The conduit porous material 1910 may inhibit twisting and/or collapse of the chamber 1946, facilitate alignment of the conduit 1900, and, as previously discussed, may decrease the need to have the inlet 1904 of the conduit 1900 positioned at or near the expected gravimetric low point of the chamber 1946.

FIG. 20 is a cross-sectional schematic of a portion of a fluid collection system 2022 that includes a conduit 2000 in fluid communication with a fluid collection assembly 2040, according to an embodiment. Except as otherwise disclosed herein, the conduit 2000 and the fluid collection assembly 2040 are the same or substantially similar to any of the conduits and fluid collection assemblies disclosed herein, respectively. The conduit 2000 includes a conduit porous material 2010 including a first portion 2034 and a second portion 2035. At least a portion of the first portion 2034 extends outwardly from the passageway 2008 and at least a portion of the second portion 2035 is disposed in the passageway 2008. The first portion 2034 may include a plurality of regions. The walls 2002 of the conduit 2000 may also include a primary branch 2039 and one or more secondary branches 2041 extending from the primary branch 2039. The plurality of regions 2034 may allow the conduit 2000 to receive bodily fluids from a plurality of different locations of the chamber 2046. In an embodiment, the plurality of regions 2034 may extend sufficiently into the chamber 2046 such that the plurality or regions enclose the sides of any penis disposed in the chamber 2046. In such an embodiment, the plurality of regions 2034 increases the likelihood that bodily fluids discharged from the penis are directly received by the conduit porous material 2010 which may allow the assembly porous material 2050 to be omitted from the fluid collection assembly 2040. The conduit porous material 2010 may inhibit twisting and/or collapse of the chamber 2046, facilitate alignment of the conduit 2000, and, as previously discussed, may decrease the need to have the inlet 2004 of the conduit 2000 positioned at or near the expected gravimetric low point of the chamber 2046.

The conduits disclosed herein may be used with fluid collection assemblies other than the fluid collection assemblies illustrated in FIG. 7A-20. For example, the conduits disclosed herein may be used with a Foley catheter, a condom-style male catheter, or wound dressing. Other examples of fluid collection assemblies that the conduits disclosed herein may be attached to are disclosed in U.S. patent application Ser. No. 16/433,773 filed on Jun. 6, 2019, the disclosure of which is incorporated herein, in its entirety, by this reference. It is also noted that at least some of the fluid collection assemblies may be used for wound care to receive one or more bodily fluids (e.g., blood, etc.) from a wound.

FIG. 21 is a block diagram of a fluid collection system 2122 for fluid collection, according to an embodiment. The system 2122 includes a fluid collection assembly 2140, a fluid storage container 2190, and a vacuum source 2192. The fluid collection assembly 2140, the fluid storage container 2190, and the vacuum source 2192 may be fluidly coupled to each other via one or more conduits. For example, as illustrated, the conduits may include a first conduit 2194a extending from the fluid collection assembly 2140 to the fluid storage container 2190 and a second conduit 2194b extending from the fluid storage container 2190 to the vacuum source 2192.

In an embodiment, the first conduit 2194a includes at least one conduit porous material. In an example, the conduit porous material may be disposed in all of the first conduit 2194a (e.g., extends from or near the inlet of the first conduit 2194a to or near the outlet of the first conduit 2194a). In an example, the conduit porous material may be disposed in only a portion of the first conduit 2194a. In an embodiment, the second conduit 2194b includes at least one conduit porous material. Similar to the first conduit 2194a, the conduit porous material of the second conduit 2194b may be disposed in all of the second conduit 2194b or only a portion of the second conduit 2194b. In an embodiment, one of the first or second conduit 2194a, 2194b may include a hollow conduit.

In an embodiment, the fluid collection system 2122 may include at least one hollow conduit (not shown) in addition to the first conduit 2194a and/or the second conduit 2194b. In an example, the first conduit 2194a may be in direct fluid communication with the fluid collection assembly 2140 and may extend from the fluid collection assembly 2140 only part of the distance between the fluid collection assembly 2140 and the fluid storage container 2190. As such, the fluid collection system 2122 may include a hollow conduit that is connected to the outlet of the first conduit 2194a and extends from the first conduit 2194a to the fluid storage container 2190.

The fluid collection assembly 2140 may be similar or identical to any of the fluid collection assemblies disclosed herein in one or more aspects. The fluid collection assembly 2140 may be shaped and sized to be positioned adjacent to a female urethral opening or have a male urethral opening positioned therethrough (e.g., receive a penis therein). For example, the fluid collection assembly 2140 may include a fluid impermeable barrier at least partially defining a chamber (e.g., interior region) of the fluid collection assembly 2140. The fluid impermeable barrier also defines at least one opening extending therethrough from the external environment. The opening may be positioned adjacent to a female urethral opening or have a male urethral opening positioned therethrough. The fluid collection assembly 2140 may include porous material disposed in the chamber such as one or more of a fluid permeable support and a fluid permeable membrane. The fluid collection assembly 2140 includes one or more of any of the securement bodies disclosed herein.

The fluid storage container 2190 is sized and shaped to retain the bodily fluids therein. The fluid storage container 2190 may include a bag (e.g., drainage bag), a bottle or cup (e.g., collection jar), or any other enclosed container for storing bodily fluids such as urine. In some examples, the first conduit 2194a may extend from the fluid collection assembly 2140 and attach to the fluid storage container 2190 at a first point therein. The second conduit 2194b may attach to the fluid storage container 2190 at a second point thereon and may extend and attach to the vacuum source 2192. Accordingly, a vacuum (e.g., suction) may be drawn through fluid collection assembly 2140 via the fluid storage container 2190. Fluid, such as urine, may be drained from the fluid collection assembly 2140 using the vacuum source 2192.

The vacuum source 2192 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 2192 may provide a vacuum or suction to remove fluid from the fluid collection assembly 2140. In some examples, the vacuum source 2192 may be powered by one or more of a power cord (e.g., connected to a power socket), one or more batteries, or even manual power (e.g., a hand operated vacuum pump). In some examples, the vacuum source 2192 may be sized and shaped to fit outside of, on, or within the fluid collection assembly 2140. For example, the vacuum source 2192 may include one or more miniaturized pumps or one or more micro pumps. The vacuum source 2192 may include one or more of a switch, a button, a plug, a remote, or any other device suitable to activate the vacuum source 2192.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, +5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A conduit for use in a fluid collection system for collecting one or more bodily fluids, the conduit comprising:
at least one wall at least partially defining at least:
an inlet;
an outlet; and
a passageway extending from the inlet to the outlet; and
at least one conduit porous material disposed in at least a portion of the passageway, the at least one conduit porous material substantially occupying a cross-sectional area of the passageway and extending at least from or near the inlet to or near the outlet.

2. The conduit of claim 1, wherein the at least one wall includes polyvinyl chloride.

3. The conduit of claim 1, wherein the at least one wall is formed from a film.

4. The conduit of claim 1, wherein the at least one conduit porous material includes spun nylon.

5. (canceled)

6. The conduit of claim 1, wherein the at least one conduit porous material is only disposed in the passageway.

7. The conduit of claim 1, wherein the at least one conduit porous material includes a first portion and a second portion, at least a portion of the first portion is not disposed in the passageway and at least a portion of the second portion is disposed in the passageway.

8. The conduit of claim 7, wherein the first portion exhibits a generally semi-spherical shape or a bulb-like shape.

9. The conduit of claim 7, wherein the first portion exhibits a maximum lateral dimension that is greater than a maximum lateral dimension of the at least one wall.

10. The conduit of claim 7, wherein the first portion includes a plurality of regions that form different and distinct flow paths for the one or more bodily fluids to flow through.

11. The conduit of claim 10, wherein the at least one wall includes a primary branch and one or more secondary branches, the passageway of the primary branch including at least a portion of the second portion of the at least one conduit porous material disposed therein, the passageway of the one or more secondary branches including a portion of one or more regions of the first portion of the at least one conduit porous material disposed therein.

12. The conduit of claim 1, wherein at least a portion of the at least one conduit porous material includes a rolled sheet.

13. The conduit of claim 1, wherein at least a portion of the at least one conduit porous material exhibits a generally circular cross-sectional shape.

14. The conduit of claim 1, wherein the conduit may exhibit a bend therein exhibiting an average radius of curvature of about 2 cm without kinking.

15. A fluid collection system, comprising:
a fluid collection assembly including:
a fluid impermeable barrier defining at least a chamber, at least one opening, and a fluid outlet;
at least one assembly porous material disposed in the chamber; and
a conduit in fluid communication with the chamber, the conduit including:
at least one wall at least partially defining at least:
an inlet;
and outlet; and
a passageway extending from the inlet to the outlet; and
at least one conduit porous material distinct from the at least one assembly porous material, the at least one conduit porous material disposed in at least a portion of the passageway, the at least one conduit porous material substantially occupying a cross-sectional area of the passageway and extending at least from or near the inlet to or near the outlet.

16. The fluid collection assembly of claim 15, wherein the fluid impermeable barrier defines a reservoir, the inlet of the conduit is positioned adjacent to the reservoir, and the conduit extends through the fluid outlet.

17. The fluid collection assembly of claim 16, wherein the fluid impermeable barrier includes a distal end region and a proximal end region opposite the distal end region, the reservoir is at least partially defined by the distal end region of the fluid impermeable barrier and the fluid outlet is at or near the distal end region of the fluid impermeable barrier.

18. The fluid collection assembly of claim 15, wherein the conduit occupies substantially all of the chamber that is not occupied by the at least one assembly porous material.

19. The fluid collection assembly of claim 15, wherein the fluid impermeable barrier defines a reservoir, the fluid impermeable barrier includes a distal end region and a proximal end region opposite the distal end region, the reservoir is at least partially defined by the distal end region of the fluid impermeable barrier and the fluid outlet is at the proximal end region of the fluid impermeable barrier.

20. The fluid collection assembly of claim 15, wherein the fluid impermeable barrier extends along a longitudinal axis and the conduit extends from the fluid outlet so that it is generally parallel to the longitudinal axis.

21. The fluid collection assembly of claim 15, wherein the fluid impermeable barrier extends along a longitudinal axis and the conduit extends from the fluid outlet at an angle that is generally non-parallel to the longitudinal axis.

22. The fluid collection assembly of claim 15, wherein the fluid impermeable barrier of the fluid collection assembly forms at least a portion of the at least one wall of the conduit.

23. The fluid collection assembly of claim 15, wherein the conduit extends along at least one lateral surface of the fluid impermeable barrier.

24. (canceled)

25. (canceled)

26. The fluid collection assembly of claim 15, further comprising an adaptor, the adaptor including a conduit portion attached to the inlet of the conduit.
